(19) United States
(12) Patent Application Publication
(10) Pub. No.: US 2026/0062437 A1
(43) Pub. Date: Mar. 5, 2026

(54) ANTIVIRAL COMPOUNDS

(71) Applicant: Regents of the University of Minnesota, Minneapolis, MN (US)

(72) Inventors: Daniel Allen Harki, Minneapolis, MN (US); Samantha Anne Kennelly, Minneapolis, MN (US); Jacob Matthew Sawyer, Minneapolis, MN (US)

(73) Assignee: Regents of the University of Minnesota, Minneapolis, MN (US)

(21) Appl. No.: 19/106,120

(22) PCT Filed: Aug. 25, 2023
(86) PCT No.: PCT/US2023/031178

§ 371 Date: Feb. 24, 2025

Related U.S. Application Data
(60) Provisional application No. 63/401,471, filed on Aug. 26, 2022.

(51) Int. Cl. C07H 19/06; A61K 31/7072; A61K 31/7076; A61K 31/708; A61P 31/14; C07H 19/16

(52) U.S. Cl. CPC C07H 19/06; A61K 31/7072; A61K 31/7076; A61K 31/708; A61P 31/14; C07H 19/16

(57) ABSTRACT

The invention provides a compound of formula I: (I) or a salt thereof, wherein R¹-R⁴ have any of the values described in the specification, as well as compositions comprising a compound of formula I. The compounds are useful as antiviral agents.

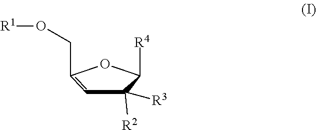

### RELATED APPLICATIONS

This application claims priority to U.S. Provisional Application No. 63/401,471 that was filed on Aug. 26, 2022. The entire content of the applications referenced above is hereby incorporated by reference herein. “

### STATEMENT REGARDING FEDERALLY SPONSORED RESEARCH

This invention was made with government support under W81XWH-21-1-0674 awarded by the Medical Research and Development Command. The government has certain rights in the invention.

### BACKGROUND OF THE INVENTION

Nucleoside analogues targeting viral polymerases are critically important therapeutics with proven efficacy against a spectrum of viruses (De Clercq, E.; Li, G., Clin. Microbiol. Rev. 2016, 29, 695). Emerging viral threats such as SARS-CoV-2, West Nile (WNV), and Zika (ZIKV) are reminders that new therapeutics are needed. 3′-Deoxy-3′,4′-didehydro-cytidine-5′-triphosphate (ddhCTP) is a recently discovered endogenous antiviral nucleotide (Gizzi, A. S. et al., Nature 2018, 558, 610). ddhCTP inhibits the polymerases of dengue (DENV), WNV, ZIKV, hepatitis C (HCV), and polio (PV, weak inhibition in competition with ribonucleoside 5′-triphosphates; Gizzi, A. S. et al., Nature 2018, 558, 610), as well as SARS-CoV-2 (Seifert, M. et al., eLife 2021, DOI: 10.7554/eLife.70968), via chain termination due to the lack of a 3′-alcohol. However, cellular studies with 3′-deoxy-3′,4′-didehydro-cytidine revealed only weak ZIKV inhibitory activity (Gizzi, A. S. et al., Nature 2018, 558, 610).

Phosphoramidate prodrugs of nucleotides, including the ProTide class of modifications (McGuigan, C. et al., Bioorg. Med. Chem. Lett. 1992, 2, 701; Mehellou, Y. et al., J. Med. Chem. 2018, 61, 2211; Alanazi, A. S. et al., ACS Med Chem Lett 2019, 10, 2; Serpi, M., Pertusati, F., Expert Opin Drug Discov 2021, 16, 1149), bypass the often rate-limiting 5′-monophosphorylation that occurs during metabolism of nucleoside drugs into their bioactive 5′-triphosphates, which enhance the efficacy of antiviral nucleosides (Murakami, E, C, et al., J. Biol. Chem. 2010, 285, 34337; Saboulard, D, C, et al., Mol. Pharmacol. 1999, 56, 693; Lee, W. A, C, et al., Antimicrob. Agents Chemother. 2005, 49, 1898; Ruane, P. J, C, et al., JAIDS 2013, 63, 449; and Siddiqui, A. Q, C, et al., J. Med. Chem. 1999, 42, 4122) and has resulted in the discoveries of sofosbuvir (Sofia, M. J, C, et al., J Med. Chem. 2010, 53, 7202) and tenofovir alafenamide (Eisenberg, E. J, C, et al., Nucleos. Nucleot. Nucl. 2001, 20, 1091; and Murakami, E, C, et al., Antimicrob. Agents Chemother. 2015, 59, 3563).

In spite of the previous studies, there remains a serious need for additional antiviral agents and treatment methods.

### SUMMARY OF THE INVENTION

Applicant has identified compounds with antiviral activity. Accordingly, the invention provides a compound of formula I:

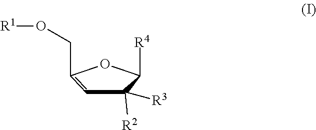

wherein:
R¹ is H, —P(═X)(OH)—O—P(═O)(OH)—P(═O)(OH)₂, or —P(═X)(R^{a})(R^{b});
R² is H, F, Cl, Br, OH, (C₁-C₃)alkyl or (C₁-C₃)alkoxy, wherein any (C₁-C₃)alkyl and (C₁-C₃)alkoxy is optionally substituted with one or more halo;
R³ is H, F, Cl, Br, OH, (C₁-C₃)alkyl or (C₁-C₃)alkoxy, wherein any (C₁-C₃)alkyl and (C₁-C₃)alkoxy is optionally substituted with one or more halo;
R⁴ is selected from the group consisting of:

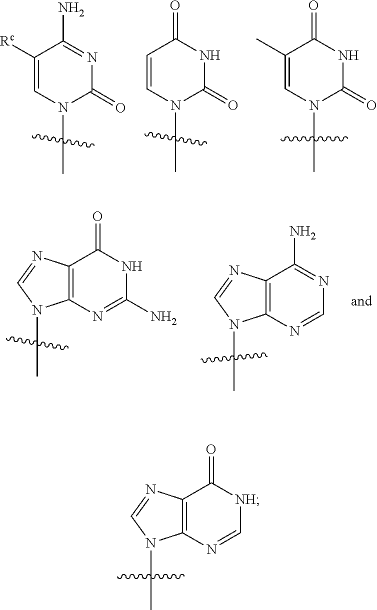

R^{a} is an N-linked amino acid or a (C₁-C₆)alkyl ester of an N-linked amino acid, wherein the (C₁-C₆)alkyl is optionally substituted with phenyl;
R^{b} is (C₁-C₆)alkoxy, aryloxy, or 5-10 membered heteroaryloxy, wherein any (C₁-C₆)alkoxy, aryloxy, and 5-10 membered heteroaryloxy is optionally substituted with one or more groups independently selected from the group consisting of halo, hydroxy, (C₁-C₃)alkoxy and (C₁-C₃)haloalkoxy;
R^{c} is (C₁-C₃)alkyl, hydroxymethyl, formyl or carboxy; and
X is S or O;
or a salt thereof.

The invention also provides a pharmaceutical composition comprising a compound of formula I or a pharmaceutically acceptable salt thereof, and a pharmaceutically acceptable excipient.

The invention also provides a method for treating or preventing a viral infection in an animal (e.g., a mammal such as a human) comprising administering a compound of formula I or a pharmaceutically acceptable salt thereof to the animal.

The invention also provides a compound of formula I or a pharmaceutically acceptable salt thereof for use in medical therapy.

The invention also provides a compound of formula I or a pharmaceutically acceptable salt thereof for the prophylactic or therapeutic treatment of a viral infection.

The invention also provides the use of a compound of formula I or a pharmaceutically acceptable salt thereof to prepare a medicament for treating a viral infection in an animal (e.g. a mammal such as a human).

The invention also provides processes and intermediates disclosed herein that are useful for preparing a compound of formula I or a salt thereof.

### BRIEF DESCRIPTION OF THE FIGURES

FIG. 1 shows cytotoxicity data for Compounds HLB-0532246 and HLB-0532247 in Vero cells from Example 5.

FIG. 2 shows cytotoxicity data for Compounds HLB-0532246 and HLB-0532247 in HUH7 cells from Example 5.

FIG. 3 shows cytotoxicity data for Compounds HLB-0535065 and HLB-0535066 in Vero Cells from Example 5.

FIG. 4 shows cytotoxicity data for Compounds HLB-0535065 and HLB-0535066 in HUH7 cells from Example 5.

FIG. 5 shows cytotoxicity data for Compounds HLB-0535069 and HLB-0532254 in Vero cells from Example 5.

FIG. 6 shows cytotoxicity data for Compounds HLB-0535069 and HLB-0532254 in HUH7 Cells from Example 5.

FIG. 7 shows cytotoxicity data for Compounds HLB-0535070 and HLB-0535071 in Vero Cells from Example 5.

FIG. 8 shows cytotoxicity data for Compounds HLB-0535070 and HLB-0535071 in HUH7 cells from Example 5.

FIG. 9 shows cytotoxicity data for Compounds HLB-0535072 and HLB-0535073 in Vero cells from Example 5.

FIG. 10 shows cytotoxicity data for Compounds HLB-0535072 and HLB-0535073 in HUH7 cells from Example 5.

FIG. 11 shows cytotoxicity data for Compound HLB-0535083 in HUH7 cells from Example 5.

FIG. 12 shows cytotoxicity data for Compound HLB-0535083 in Vero Cells from Example 5.

FIG. 13 shows antiviral activity from Example 5 for compound HLB-0535071 against West Nile Virus.

FIG. 14 shows antiviral activity from Example 5 for compound HLB-0535070 against West Nile Virus.

FIG. 15 shows antiviral activity from Example 5 for compound HLB-0535071 against Zika Virus.

FIG. 16 shows antiviral activity from Example 5 for compound HLB-0535070 against Zika Virus.

FIG. 17 shows antiviral activity from Example 5 for compound HLB-0532254 against Zika Virus.

FIG. 18 shows antiviral activity from Example 5 for compound HLB-0535072 against Zika Virus.

FIG. 19 shows antiviral activity from Example 5 for compound HLB-0535073 against Zika Virus.

### DETAILED DESCRIPTION OF THE INVENTION

The following definitions are used, unless otherwise described: halo or halogen is fluoro, chloro, bromo, or iodo. Alkyl, alkoxy, etc. denote both straight and branched groups; but reference to an individual radical such as propyl embraces only the straight chain radical, a branched chain isomer such as isopropyl being specifically referred to.

The term “alkyl”, by itself or as part of another substituent, means, unless otherwise stated, a straight or branched chain hydrocarbon radical, having the number of carbon atoms designated (i.e., C₁-C₆ means one to six carbons). Examples include (C₁-C₆)alkyl, (C₂-C₆)alkyl and (C₃-C₆)alkyl. Examples of alkyl groups include methyl, ethyl, n-propyl, iso-propyl, n-butyl, t-butyl, iso-butyl, sec-butyl, n-pentyl, and n-hexyl.

The term “alkoxy” refers to an alkyl groups attached to the remainder of the molecule via an oxygen atom (“oxy”).

The term “cycloalkyl” refers to a saturated or partially unsaturated (non-aromatic) all carbon ring having 3 to 8 carbon atoms (i.e., (C₃-C₈)carbocycle). The term also includes multiple condensed, saturated all carbon ring systems (e.g., ring systems comprising 2, 3 or 4 carbocyclic rings). Accordingly, carbocycle includes multicyclic carbocycles such as a bicyclic carbocycles (e.g., bicyclic carbocycles having about 3 to 15 carbon atoms, about 6 to 15 carbon atoms, or 6 to 12 carbon atoms such as bicyclo[3.1.0]hexane and bicyclo[2.1.1]hexane), and polycyclic carbocycles (e.g tricyclic and tetracyclic carbocycles with up to about 20 carbon atoms). The rings of the multiple condensed ring system can be connected to each other via fused, spiro and bridged bonds when allowed by valency requirements. For example, multicyclic carbocycles can be connected to each other via a single carbon atom to form a spiro connection (e.g., spiropentane, spiro[4,5]decane, etc), via two adjacent carbon atoms to form a fused connection (e.g., carbocycles such as decahydronaphthalene, norsabinane, norcarane) or via two non-adjacent carbon atoms to form a bridged connection (e.g., norbornane, bicyclo[2.2.2]octane, etc). Non-limiting examples of cycloalkyls include cyclopropyl, cyclobutyl, cyclopentyl, cyclohexyl, bicyclo[2.2.1]heptane, pinane, and adamantane.

The term “aryl” as used herein refers to a single all carbon aromatic ring or a multiple condensed all carbon ring system wherein at least one of the rings is aromatic. For example, in certain embodiments, an aryl group has 6 to 20 carbon atoms, 6 to 14 carbon atoms, 6 to 12 carbon atoms, or 6 to 10 carbon atoms. Aryl includes a phenyl radical. Aryl also includes multiple condensed carbon ring systems (e.g., ring systems comprising 2, 3 or 4 rings) having about 9 to 20 carbon atoms in which at least one ring is aromatic and wherein the other rings may be aromatic or not aromatic (i.e., cycloalkyl). The rings of the multiple condensed ring system can be connected to each other via fused, spiro and bridged bonds when allowed by valency requirements. It is to be understood that the point of attachment of a multiple condensed ring system, as defined above, can be at any position of the ring system including an aromatic or a carbocycle portion of the ring. Non-limiting examples of aryl groups include, but are not limited to, phenyl, indenyl, indanyl, naphthyl, 1, 2, 3, 4-tetrahydronaphthyl, anthracenyl, and the like.

The term “heterocycle” refers to a single saturated or partially unsaturated ring that has at least one atom other than carbon in the ring, wherein the atom is selected from the group consisting of oxygen, nitrogen and sulfur; the term also includes multiple condensed ring systems that have at least one such saturated or partially unsaturated ring, which multiple condensed ring systems are further described below. Thus, the term includes single saturated or partially unsaturated rings (e.g., 3, 4, 5, 6 or 7-membered rings) from about 1 to 6 carbon atoms and from about 1 to 3 heteroatoms selected from the group consisting of oxygen, nitrogen and sulfur in the ring. The sulfur and nitrogen atoms may also be present in their oxidized forms. Exemplary heterocycles include but are not limited to azetidinyl, tetrahydrofuranyl and piperidinyl. The term “heterocycle” also includes multiple condensed ring systems (e.g., ring systems comprising 2, 3 or 4 rings) wherein a single heterocycle ring (as defined above) can be condensed with one or more groups selected from cycloalkyl, aryl, and heterocycle to form the multiple condensed ring system. The rings of the multiple condensed ring system can be connected to each other via fused, spiro and bridged bonds when allowed by valency requirements. It is to be understood that the individual rings of the multiple condensed ring system may be connected in any order relative to one another. It is also to be understood that the point of attachment of a multiple condensed ring system (as defined above for a heterocycle) can be at any position of the multiple condensed ring system including a heterocycle, aryl and carbocycle portion of the ring. In one embodiment the term heterocycle includes a 3-15 membered heterocycle. In one embodiment the term heterocycle includes a 3-10 membered heterocycle. In one embodiment the term heterocycle includes a 3-8 membered heterocycle. In one embodiment the term heterocycle includes a 3-7 membered heterocycle. In one embodiment the term heterocycle includes a 3-6 membered heterocycle. In one embodiment the term heterocycle includes a 4-6 membered heterocycle. In one embodiment the term heterocycle includes a 3-10 membered monocyclic or bicyclic heterocycle comprising 1 to 4 heteroatoms. In one embodiment the term heterocycle includes a 3-8 membered monocyclic or bicyclic heterocycle comprising 1 to 3 heteroatoms. In one embodiment the term heterocycle includes a 3-6 membered monocyclic heterocycle comprising 1 to 2 heteroatoms. In one embodiment the term heterocycle includes a 4-6 membered monocyclic heterocycle comprising 1 to 2 heteroatoms. Exemplary heterocycles include, but are not limited to aziridinyl, azetidinyl, pyrrolidinyl, piperidinyl, homopiperidinyl, morpholinyl, thiomorpholinyl, piperazinyl, tetrahydrofuranyl, dihydrooxazolyl, tetrahydropyranyl, tetrahydrothiopyranyl, 1,2,3,4-tetrahydroquinolyl, benzoxazinyl, dihydrooxazolyl, chromanyl, 1,2-dihydropyridinyl, 2,3-dihydrobenzofuranyl, 1,3-benzodioxolyl, 1,4-benzodioxanyl, spiro[cyclopropane-1,1′-isoindolinyl]-3′-one, isoindolinyl-1-one, 2-oxa-6-azaspiro[3.3]heptanyl, imidazolidin-2-one imidazolidine, pyrazolidine, butyrolactam, valerolactam, imidazolidinone, hydantoin, dioxolane, phthalimide, and 1,4-dioxane.

The term “heteroaryl” as used herein refers to a single aromatic ring that has at least one atom other than carbon in the ring, wherein the atom is selected from the group consisting of oxygen, nitrogen and sulfur; “heteroaryl” also includes multiple condensed ring systems that have at least one such aromatic ring, which multiple condensed ring systems are further described below. Thus, “heteroaryl” includes single aromatic rings of from about 1 to 6 carbon atoms and about 1-4 heteroatoms selected from the group consisting of oxygen, nitrogen and sulfur. The sulfur and nitrogen atoms may also be present in an oxidized form provided the ring is aromatic. Exemplary heteroaryl ring systems include but are not limited to pyridyl, pyrimidinyl, oxazolyl or furyl. “Heteroaryl” also includes multiple condensed ring systems (e.g., ring systems comprising 2, 3 or 4 rings) wherein a heteroaryl group, as defined above, is condensed with one or more rings selected from cycloalkyl, aryl, heterocycle, and heteroaryl. It is to be understood that the point of attachment for a heteroaryl or heteroaryl multiple condensed ring system can be at any suitable atom of the heteroaryl or heteroaryl multiple condensed ring system including a carbon atom and a heteroatom (e.g., a nitrogen). Exemplary heteroaryls include but are not limited to pyridyl, pyrrolyl, pyrazinyl, pyrimidinyl, pyridazinyl, pyrazolyl, thienyl, indolyl, imidazolyl, triazolyl, tetrazolyl, oxazolyl, isoxazolyl, thiazolyl, furyl, oxadiazolyl, thiadiazolyl, quinolyl, isoquinolyl, benzothiazolyl, benzoxazolyl, indazolyl, quinoxalyl, and quinazolyl.

As used herein, the term “heteroatom” is meant to include oxygen (O), nitrogen (N), sulfur (S) and silicon (Si).

The term “amino acid,” comprises the natural amino acids (e.g. Ala, Arg, Asn, Asp, Cys, Glu, Gln, Gly, His, Hyl, Hyp, Ile, Leu, Lys, Met, Phe, Pro, Ser, Thr, Trp, Tyr, and Val) in D or L form, as well as unnatural amino acids (e.g. phosphoserine, phosphothreonine, phosphotyrosine, hydroxyproline, gamma-carboxyglutamate; hippuric acid, octahydroindole-2-carboxylic acid, statine, 1,2,3,4,-tetrahydroisoquinoline-3-carboxylic acid, penicillamine, ornithine, citruline, α-methyl-alanine, para-benzoylphenylalanine, phenylglycine, propargylglycine, sarcosine, and tert-butylglycine).

As used herein, the term “protecting group” refers to a substituent that is commonly employed to block or protect a particular functional group on a compound. For example, an “amino-protecting group” is a substituent attached to an amino group that blocks or protects the amino functionality in the compound. Suitable amino-protecting groups include acetyl, trifluoroacetyl, t-butoxycarbonyl (BOC), benzyloxycarbonyl (CBZ) and 9-fluorenylmethylenoxycarbonyl (Fmoc). Similarly, a “hydroxy-protecting group” refers to a substituent of a hydroxy group that blocks or protects the hydroxy functionality. Suitable protecting groups include acetyl and silyl. A “carboxy-protecting group” refers to a substituent of the carboxy group that blocks or protects the carboxy functionality. Common carboxy-protecting groups include phenylsulfonylethyl, cyanoethyl, 2-(trimethylsilyl)ethyl, 2-(trimethylsilyl)ethoxymethyl, 2-(p-toluenesulfonyl)ethyl, 2-(p-nitrophenylsulfenyl)ethyl, 2-(diphenylphosphino)-ethyl, nitroethyl and the like. For a general description of protecting groups and their use, see P. G. M. Wuts and T. W. Greene, Greene's Protective Groups in Organic Synthesis 4^{th} edition, Wiley-Interscience, New York, 2006.

As used herein a wavy line “ [symbol] ” that intersects a bond in a chemical structure indicates the point of attachment of the bond that the wavy bond intersects in the chemical structure to the remainder of a molecule.

The terms “treat”, “treatment”, or “treating” to the extent it relates to a disease or condition includes inhibiting the disease or condition, eliminating the disease or condition, and/or relieving one or more symptoms of the disease or condition. The terms “treat”, “treatment”, or “treating” also refer to both therapeutic treatment and/or prophylactic treatment or preventative measures, wherein the object is to prevent or slow down (lessen) an undesired physiological change or disorder, such as, for example, the development or spread of cancer. For example, beneficial or desired clinical results include, but are not limited to, alleviation of symptoms, diminishment of extent of disease or disorder, stabilized (i.e., not worsening) state of disease or disorder, delay or slowing of disease progression, amelioration or palliation of the disease state or disorder, and remission (whether partial or total), whether detectable or undetectable. “Treat”, “treatment”, or “treating,” can also mean prolonging survival as compared to expected survival if not receiving treatment. Those in need of treatment include those already with the disease or disorder as well as those prone to have the disease or disorder or those in which the disease or disorder is to be prevented. In one embodiment “treat”, “treatment”, or “treating” does not include preventing or prevention,

The phrase “therapeutically effective amount” or “effective amount” includes but is not limited to an amount of a compound of the that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein.

The term “mammal” as used herein refers to humans, higher non-human primates, rodents, domestic, cows, horses, pigs, sheep, dogs and cats. In one embodiment, the mammal is a human. The term “patient” as used herein refers to any animal including mammals. In one embodiment, the patient is a mammalian patient. In one embodiment, the patient is a human patient.

The compounds disclosed herein can also exist as tautomeric isomers in certain cases. Although only one delocalized resonance structure may be depicted, all such forms are contemplated within the scope of the invention.

It is understood by one skilled in the art that this invention also includes any compound claimed that may be enriched at any or all atoms above naturally occurring isotopic ratios with one or more isotopes such as, but not limited to, deuterium (²H or D). As a non-limiting example, a —CH₃ group may be substituted with —CD₃.

The pharmaceutical compositions of the invention can comprise one or more excipients. When used in combination with the pharmaceutical compositions of the invention the term “excipients” refers generally to an additional ingredient that is combined with the compound of formula (I) or the pharmaceutically acceptable salt thereof to provide a corresponding composition. For example, when used in combination with the pharmaceutical compositions of the invention the term “excipients” includes, but is not limited to: carriers, binders, disintegrating agents, lubricants, sweetening agents, flavoring agents, coatings, preservatives, and dyes.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., “Stereochemistry of Organic Compounds”, John Wiley & Sons, Inc., New York, 1994. The compounds of the invention can contain asymmetric or chiral centers, and therefore exist in different stereoisomeric forms. It is intended that all stereoisomeric forms of the compounds of the invention, including but not limited to, diastereomers, enantiomers and atropisomers, as well as mixtures thereof such as racemic mixtures, form part of the present invention. Many organic compounds exist in optically active forms, i.e., they have the ability to rotate the plane of plane-polarized light. In describing an optically active compound, the prefixes D and L, or R and S, are used to denote the absolute configuration of the molecule about its chiral center(s). The prefixes d and 1 or (+) and (−) are employed to designate the sign of rotation of plane-polarized light by the compound, with (−) or 1 meaning that the compound is levorotatory. A compound prefixed with (+) or d is dextrorotatory. For a given chemical structure, these stereoisomers are identical except that they are mirror images of one another. A specific stereoisomer can also be referred to as an enantiomer, and a mixture of such isomers is often called an enantiomeric mixture. A 50:50 mixture of enantiomers is referred to as a racemic mixture or a racemate, which can occur where there has been no stereoselection or stereospecificity in a chemical reaction or process. The terms “racemic mixture” and “racemate” refer to an equimolar mixture of two enantiomeric species, devoid of optical activity.

It will be appreciated by those skilled in the art that compounds of the invention having a chiral center may exist in and be isolated in optically active and racemic forms. Some compounds may exhibit polymorphism. It is to be understood that the present invention encompasses any racemic, optically-active, polymorphic, or stereoisomeric form, or mixtures thereof, of a compound of the invention, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase.

When a bond in a compound formula herein is drawn in a non-stereochemical manner (e.g. flat), the atom to which the bond is attached includes all stereochemical possibilities. When a bond in a compound formula herein is drawn in a defined stereochemical manner (e.g. bold, bold-wedge, dashed or dashed-wedge), it is to be understood that the atom to which the stereochemical bond is attached is enriched in the absolute stereoisomer depicted unless otherwise noted. In one embodiment, the compound may be at least 51% the absolute stereoisomer depicted. In another embodiment, the compound may be at least 60% the absolute stereoisomer depicted. In another embodiment, the compound may be at least 80% the absolute stereoisomer depicted. In another embodiment, the compound may be at least 90% the absolute stereoisomer depicted. In another embodiment, the compound may be at least 95 the absolute stereoisomer depicted. In another embodiment, the compound may be at least 99% the absolute stereoisomer depicted.

Specific values listed below for radicals, substituents, and ranges, are for illustration only; they do not exclude other defined values or other values within defined ranges for the radicals and substituents. It is to be understood that two or more values may be combined. It is also to be understood that the values listed herein below (or subsets thereof) can be excluded.

Specifically, (C₁-C₆)alkyl can be methyl, ethyl, propyl, isopropyl, butyl, iso-butyl, sec-butyl, pentyl, 3-pentyl, or hexyl; (C₁-C₆)alkoxy can be methoxy, ethoxy, propoxy, isopropoxy, butoxy, iso-butoxy, sec-butoxy, pentoxy, 3-pentoxy, or hexyloxy; aryl can be phenyl, indenyl, or naphthyl; and heteroaryl can be furyl, imidazolyl, triazolyl, triazinyl, oxazoyl, isoxazoyl, thiazolyl, isothiazoyl, pyrazolyl, pyrrolyl, pyrazinyl, tetrazolyl, pyridyl, (or its N-oxide), thienyl, pyrimidinyl (or its N-oxide), indolyl, isoquinolyl (or its N-oxide) or quinolyl (or its N-oxide).

A specific value for R¹ is H.

A specific value for R¹ is —P(═O)(OH)—O—P(═O)(OH)—P(═O)(OH)₂.

or —P(═O)(R^{a})(R^{b}).

A specific value for R² is F, Cl, Br, OH, (C₁-C₃)alkyl or (C₁-C₃)alkoxy, wherein any (C₁-C₃)alkyl and (C₁-C₃)alkoxy is optionally substituted with one or more (e.g., 1, 2, 3, or 4) halo.

A specific value for R² is H.

A specific value for R² is F, OH, or methoxy.

A specific value for R² is OH.

A specific value for R³ is F, Cl, Br, OH, (C₁-C₃)alkyl or (C₁-C₃)alkoxy, wherein any (C₁-C₃)alkyl and (C₁-C₃)alkoxy is optionally substituted with one or more (e.g., 1, 2, 3, or 4) halo.

A specific value for R³ is H.

A specific value for R³ is methyl or F.

A specific value for R⁴ is selected from the group consisting of:

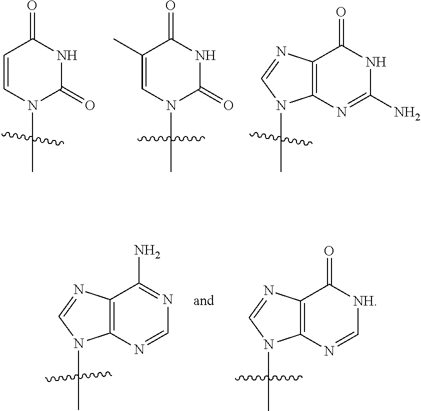

A specific value for R⁴ is:

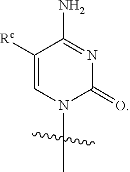

A specific value for R⁴ is:

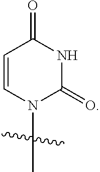 [SMILES: *n1ccc(=O)[nH]c1=O]

A specific value for R⁴ is:

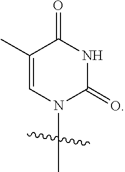

A specific value for R⁴ is:

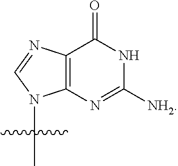 [SMILES: *n1cnc2c(=O)[nH]c(N)nc21]

A specific value for R⁴ is:

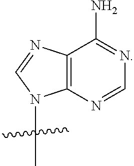

A specific value for R⁴ is:

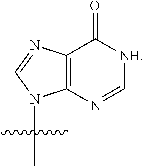

A specific value for R^{a} is an N-linked amino acid or a (C₁-C₆)alkyl ester of an N-linked amino acid.

A specific value for R^{a} is a (C₁-C₆)alkyl ester of an N-linked amino acid, wherein the (C₁-C₆)alkyl is optionally substituted with phenyl.

A specific value for R^{a} is a (C₁-C₆)alkyl ester of an N-linked amino acid.

A specific value for R^{a} is R^{d}—O—C(═O)—CH(R^{e})—NH—; wherein R^{d} is (C₁-C₆)alkyl that is optionally substituted with phenyl; and R^{e} is the side-chain of a natural amino acid.

A specific value for R^{b} is (C₁-C₆)alkoxy that is optionally substituted with one or more groups independently selected from the group consisting of halo, hydroxy, and (C₁-C₃)alkoxy.

A specific value for R^{b} is aryloxy that is optionally substituted with one or more groups independently selected from the group consisting of halo, hydroxy, (C₁-C₃)alkoxy and (C₁-C₃)haloalkoxy.

A specific value for R^{b} is 5-10 membered heteroaryloxy that is optionally substituted with one or more groups independently selected from the group consisting of halo, hydroxy, (C₁-C₃)alkoxy and (C₁-C₃)haloalkoxy.

A specific value for R^{b} is phenoxy that is optionally substituted with one or more groups independently selected from the group consisting of halo, hydroxy, (C₁-C₃)alkoxy and (C₁-C₃)haloalkoxy.

A specific value for R^{b} is phenoxy.

A specific value for R^{c} is methyl, hydroxymethyl, formyl or carboxy.

A specific value for R^{d} is methyl, ethyl, isopropyl, n-butyl, tert-butyl, or benzyl.

A specific value for R^{d} is isopropyl.

A specific value for R^{e} is H or methyl.

A specific value for R^{e} is methyl.

A specific compound of formula (I) is a compound of formula (Ia):

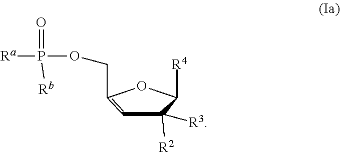

A specific compound of formula (I) is a compound of formula (Ib):

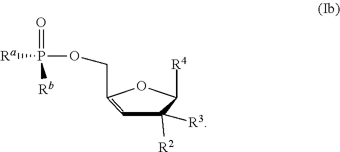

A specific compound of formula (I) is a compound of formula (Ic):

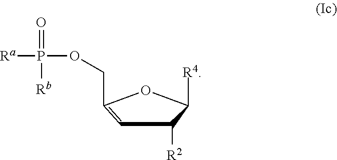

A specific compound of formula (I) is a compound of formula (Id):

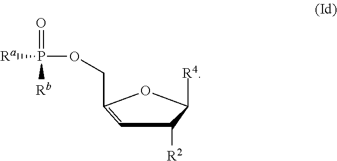

A specific value for X is S.

A specific value for X is O.

In cases where compounds are sufficiently basic or acidic, a salt of a compound of formula I can be useful as an intermediate for isolating or purifying a compound of formula I. Additionally, administration of a compound of formula I as a pharmaceutically acceptable acid or base salt may be appropriate. Examples of pharmaceutically acceptable salts are organic acid addition salts formed with acids which form a physiological acceptable anion, for example, tosylate, methanesulfonate, acetate, citrate, malonate, tartarate, succinate, benzoate, ascorbate, α-ketoglutarate, and α-glycerophosphate. Suitable inorganic salts may also be formed, including hydrochloride, sulfate, nitrate, bicarbonate, and carbonate salts.

Salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

The compounds of formula I can be formulated as pharmaceutical compositions and administered to a mammalian host, such as a human patient in a variety of forms adapted to the chosen route of administration, i.e., orally or parenterally, by intravenous, intramuscular, topical or subcutaneous routes.

Thus, the present compounds may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound may also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present compounds may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver the compounds of formula I to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful dosages of the compounds of formula I can be determined by comparing their in vitro activity, and in vivo activity in animal models. Methods for the extrapolation of effective dosages in mice, and other animals, to humans are known to the art; for example, see U.S. Pat. No. 4,938,949.

The amount of the compound, or an active salt or derivative thereof, required for use in treatment will vary not only with the particular salt selected but also with the route of administration, the nature of the condition being treated and the age and condition of the patient and will be ultimately at the discretion of the attendant physician or clinician.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

Compounds of the invention can also be administered in combination with other therapeutic agents, for example, other agents that are useful for the treatment of viral infections. Examples of such agents include:
HIV protease inhibitors, such as, for example, saquinavir, ritonavir, indinavir, nelfinavir, lopinavir, atazanavir, fosamprenavir, tipranavir, and darunavir;
HIV integrase inhibitors, such as, for example, raltegravir, elvitegravir, dolutegravir, and bictegravir;
HIV receptor inhibitors, such as, for example, enfuvirtide, albuvirtide; and
Biologics, such as, for example, convalescent antibodies and monoclonal antibodies. For additional information see DOI: 10.1039/9781788016858-00001

Accordingly, in one embodiment the invention also provides a composition comprising a compound of formula I, or a pharmaceutically acceptable salt thereof, at least one other therapeutic agent, and a pharmaceutically acceptable diluent or carrier. The invention also provides a kit comprising a compound of formula I, or a pharmaceutically acceptable salt thereof, at least one other therapeutic agent, packaging material, and instructions for administering the compound of formula I or the pharmaceutically acceptable salt thereof and the other therapeutic agent or agents to an animal to treat a viral infection.

The antiviral activity of a compound can be determined using pharmacological models which are well known to the art, or using the models described herein.

The invention will now be illustrated by the following non-limiting Examples.

### EXAMPLES

### Example 1. Synthesis of Isopropyl ((S)-(((4R,5R)-5-(2,4-dioxo-3,4-dihydropyrimidin-1(2H)-yl)-4-hydroxy-4,5-dihydrofuran-2-yl)methoxy)(phenoxy)phosphoryl)-L-alaninate

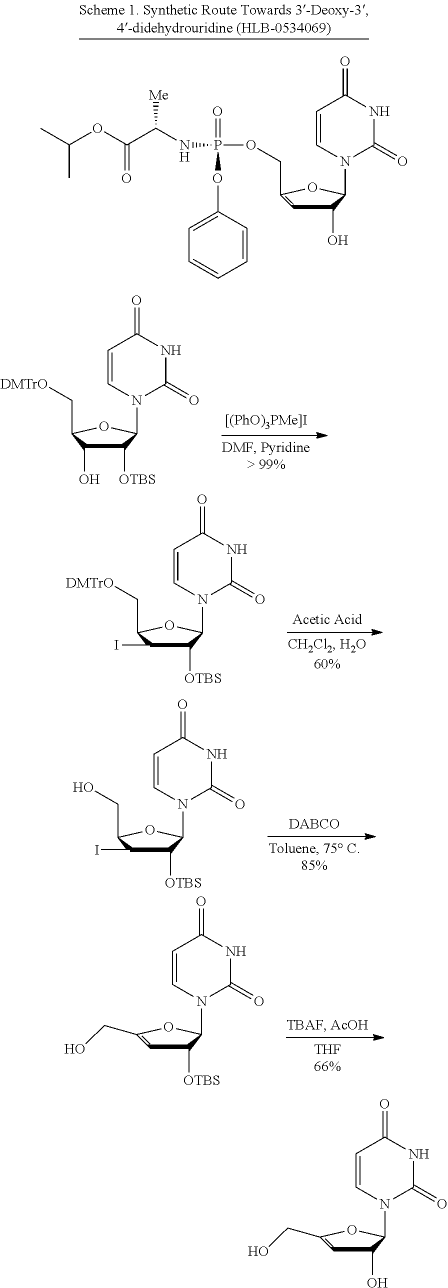

### a. 5′-DMT-3′-Iodo-2′-TBS-uridine

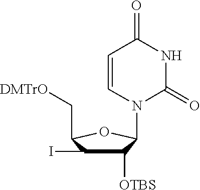

5′-DMT-2′-TBS-uridine (Carbosynth, NB15303; 1.01 g, 1.0 mmol) was placed in a Schlenk flask and placed on the high-vacuum for one hour before the addition of methyltriphenoxyphosphonium iodide (1.10 g, 1.61 mmol). The flask was then purged and filled with nitrogen 3 times before the addition of solvent. To the purged flask was added DMF (5 mL) and then pyridine (241 μL, 1.98 mmol). The reaction was stirred under nitrogen overnight (18 hours). The flask was covered in aluminum foil as the [(PhO)₃PMe]I reagent is light sensitive. After 18 hours, the reaction was quenched by the addition of CH₃OH (1 mL) and TEA (1 mL) and stirred for 15 minutes. The aqueous layer was extracted using ethyl acetate (20 mL) and washed with water 2× (20 mL) and brine 1× (20 mL). The organics were dried over sodium sulfate and concentrated in vacuo. The crude material was purified via flash column chromatography using hexanes:EtOAc from 0-30% over 20 minutes and a final push to 100% EtOAc for 5 minutes. 5′-DMT-3′-Iodo-2′-TBS-uridine eluted in 30% EtOAc in hexanes and formed a yellow foam after concentrating in vacuo (1.21 g, >99%). ¹H NMR (500 MHz, DMSO-d₆): 11.47 (s, 1H), 7.52 (d, J=8.1 Hz, 1H), 7.45-7.39 (m, 2H), 7.37-7.30 (m, 3H), 7.30-7.23 (m, 4H), 6.94-6.88 (m, 4H), 5.63 (d, J=3.5 Hz, 1H), 5.39 (d, J=8.1 Hz, 1H), 4.64 (t, J=3.8 Hz, 1H), 4.40 (dd, J=5.9, 4.2 Hz, 1H), 4.16-4.09 (m, 1H), 3.74 (m, J=1.1 Hz, 6H), 3.46-3.39 (m, 1H), 3.32-3.28 (m, 1H), 0.87 (s, 9H), 0.14 (s, 3H), 0.05 (s, 3H) δ ppm. ¹³C NMR (151 MHz, DMSO-d₆): 162.9, 158.2 (2C), 150.4, 144.4, 139.9, 135.1, 134.9, 129.9 (2C), 129.8 (2C), 127.8 (2C), 127.8 (2C), 126.9, 113.2 (4C), 101.1, 89.9, 86.2, 82.3, 78.7, 68.6, 55.0 (2C), 27.9, 25.4 (3C), 17.5, −4.5, −5.0 δ ppm. HRMS (ESI) m/z: calcd for C₃₆H₄₃IN₂O₇Si [M-H]⁻: 769.1811; found 769.1789.

### b. 3′-Iodo-2′-TBS-uridine

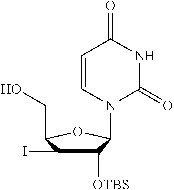

5′-DMT-3′-Iodo-2′-TBS-uridine (1.21 g, 1.0 mmol) was dissolved in CH₂Cl₂ (10 mL) and to this solution was added a 4:1 mixture of acetic acid:water (10 mL:2.5 mL). Upon the addition of the acetic acid:water mixture, the reaction solution immediately turned orange in color. The reaction mixture was stirred at RT overnight. The reaction was then adjusted to a pH of 7 by the addition of saturated aqueous NaHCO₃ and the reaction was extracted in EtOAc (30 mL). The organic layer was washed with water 2× (30 mL) and brine 1× (30 mL). The collected organic layer was then dried over Na₂SO₄, filtered and concentrated in vacuo. The crude compound was purified on silica gel using hexanes:EtOAc up to 100% EtOAc. 3′-Iodo-2′-TBS-uridine eluted in 60% EtOAc in hexanes and formed a yellow foam after concentrating in vacuo (590 mg, 80%). ¹H NMR (500 MHz, DMSO-d₆): 11.43 (s, 1H), 7.93 (d, J=8.2 Hz, 1H), 5.73 (d, J=8.1 Hz, 1H), 5.67 (d, J=4.6 Hz, 1H), 5.21 (t, J=4.5 Hz, 1H), 4.61 (t, J=5.2 Hz, 1H), 4.45 (t, J=6.2 Hz, 1H), 4.04-3.98 (m, 1H), 3.80-3.73 (m, 1H), 3.72-3.65 (m, 1H), 0.85 (s, 9H), 0.12 (s, 3H), 0.00 (s, 3H) δ ppm. ¹³C NMR (151 MHz, DMSO-d₆): 163.1, 150.7, 140.5, 102.1, 88.6, 81.5, 79.7, 65.6, 28.1, 25.6 (3C), 17.7, −4.2, −4.9 δ ppm. HRMS (ESI) m/z: calcd for C₁₅H₂₅IN₂O₅Si [M-H]⁻: 467.0505; found 467.0489.

### c. 3′-Deoxy-3′,4′-didehydro-2′-TBS uridine

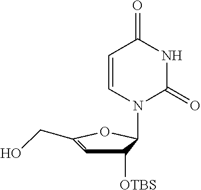 [SMILES: CC(C)(C)[Si](C)(C)O[C@H]1C=C(CO)O[C@H]1n1ccc(=O)[nH]c1=O]

3′-Iodo-2′-TBS-uridine (590 mg, 1.0 mmol) was dissolved in toluene (10 mL) and to the solution was added DABCO (495 mg, 1.26 mmol). The reaction mixture was then heated to 75° C. and stirred overnight (18 hours). The next day, the reaction mixture was cooled and filtered to remove the salts formed. The collected filtrate was concentrated in vacuo and purified via flash chromatography using CH₃OH:CH₂Cl₂ from 0-25% over 20 minutes. 3′-Deoxy-3′,4′-didehydro-2′-TBS uridine eluted in 10% CH₃OH in CH₂Cl₂ and formed a yellow foam after concentrating in vacuo (366 mg, 85%). ¹H NMR (500 MHz, DMSO-d₆): 11.47 (s, 1H), 7.29 (d, J=8.0 Hz, 1H), 6.10 (d, J=2.2 Hz, 1H), 5.65 (d, J=8.0 Hz, 1H), 5.32 (t, J=5.9 Hz, 1H), 5.17-5.15 (m, 1H), 5.01-4.97 (m, 1H), 4.05 (d, J=5.0 Hz, 2H), 0.85 (s, 9H), 0.06 (d, J=9.8 Hz, 6H) δ ppm. 13C NMR (151 MHz, DMSO-d₆): 163.2, 162.4, 150.3, 139.9, 102.7, 100.2, 92.6, 79.6, 56.1, 25.7 (3C), 17.8, −4.5, −4.6 δ ppm. HRMS (ESI) m/z: calcd for C₁₅H₂₄N₂O₅Si [M-H]⁻: 339.1382; found 339.1379.

### d. 3′-Deoxy-3′,4′-didehydrouridine

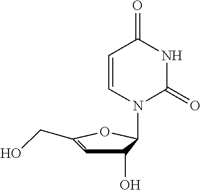 [SMILES: O=c1ccn([C@@H]2OC(CO)=C[C@@H]2O)c(=O)[nH]1]

3′-Deoxy-3′,4′-didehydro-2′-TBS uridine (366 mg, 1.0 mmol) was dissolved in THF. To this was added a 1:1 mixture of acetic acid:TBAF (1M in THF; 1.48 mmol). The reaction stirred overnight. The next day, the reaction was concentrated in vacuo and purified via flash chromatography using CH₃OH:CH₂Cl₂ from 0-25% over 20 minutes. 3′-Deoxy-3′,4′-didehydrouridine eluted in 10% CH₃OH in CH₂Cl₂ and formed a white foam after concentrating in vacuo (210 mg, 86%). ¹H NMR (500 MHz, CD₃OD): 7.77 (d, J=8.1 Hz, 1H), 6.19 (s, 1H), 5.84-5.78 (m, 2H), 5.20 (dd, J=6.5, 2.5 Hz, 1H), 5.01 (dd, J=6.5, 4.0 Hz, 1H), 4.31-4.25 (m, 1H), 3.88 (dd, J=12.3, 3.9 Hz, 1H), 3.81 (dd, J=12.3, 5.7 Hz, 1H) δ ppm. ¹³C NMR (151 MHz, CD₃OD): 164.0, 148.9, 141.0, 115.0, 99.4, 90.4, 83.5, 80.9, 77.6, 58.8 δ ppm. HRMS (ESI) m/z: calcd for C₉H₁₀N₂O₅ [M-H]⁻: 225.0517; found 225.0510.

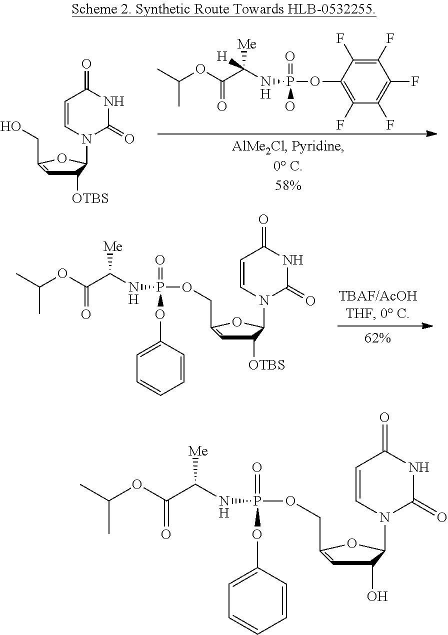

### e. Isopropyl ((S)-(((4R,5R)-4-((tert-butyldimethylsilyl)oxy)-5-(2,4-dioxo-3,4-dihydropyrimidin-1(2H)-yl)-4,5-dihydrofuran-2-yl)methoxy)(phenoxy)phosphoryl)-L-alaninate

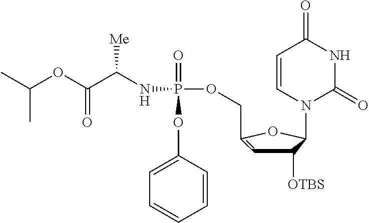 [SMILES: CC(C)OC(=O)[C@H](C)N[P@](=O)(OCC1=C[C@H](O[Si](C)(C)C(C)(C)C)[C@H](n2ccc(=O)[nH]c2=O)O1)Oc1ccccc1]

3′-Deoxy-3′,4′-didehydro-2′-TBS uridine (220 mg, 1.0 mmol) and propan-2-yl (2S)-2-{[(2,3,4,5,6 pentafluorophenoxy)(phenoxy)phosphoryl]amino}propanoate (439 mg, 1.5 mmol) were placed in a Schlenk flask equipped with a stir bar and was dried overnight under vacuum using a needle and septa (placed on the high-vacuum). The next morning, the Schlenk flask was purged with nitrogen and anhydrous pyridine was added (6 mL). The reaction was then cooled to 0° C. using an ice bath and stirred for 20 minutes before the dropwise addition of dimethylaluminum chloride (1 M in hexanes, 323 μL, 0.5 mmol). The reaction was stirred at 0° C. for 30 minutes before removing the ice bath and letting the reaction warm-up to RT. The reaction was stirred at RT for 48 hours. After 48 hours, the reaction was quenched by 30% w/v aq. tartaric acid (1 mL) at 0° C. for 1 min. The reaction was then returned to RT and mixed for 5 minutes. The solution was then extracted with EtOAc (5 mL, ×3) and washed with brine (10 mL, ×2). The collected organic layers were then dried over Na₂SO₄, filtered, and the filtrate was concentrated in vacuo. The crude material was purified via column chromatography from 0-100% EtOAc:hexanes over 30 minutes. The desired material eluted at 80% EtOAc in hexanes as a white foam after concentrating in vacuo (230 mg, 58%). ¹H NMR (500 MHz, DMSO-d₆): 11.50 (bs, 1H), 7.39-7.31 (m, 3H), 7.24-7.16 (m, 3H), 6.13 (d, J=2.5 Hz, 1H), 6.10 (dd, J=13.3, 10.0 Hz, 1H), 5.61 (d, J=8.0 Hz, 1H), 5.35-5.31 (m, 1H), 5.10-5.05 (m, 1H), 4.87 (p, J=6.3 Hz, 1H), 4.68-4.59 (m, 2H), 3.83-3.74 (m, 1H), 1.25-1.19 (m, 3H), 1.19-1.12 (m, 6H), 0.85 (s, 9H), 0.06 (d, J=6.7 Hz, 6H) δ ppm. ¹³C NMR (126 MHz, DMSO-d₆): 172.5 (d, J_{C-P}=4.9 Hz, 1C), 162.9, 156.4 (d, J_{C-P}=9.0 Hz, 1C), 150.6 (d, J_{C-P}=6.6 Hz, 1C), 150.0, 140.0, 129.5 (2C), 124.6, 120.1 (d, J_{C-P}=4.8 Hz, 2C), 103.3, 102.6, 92.8, 79.3, 67.9, 59.8 (d, J_{C-P}=4.1 Hz, 1C) 49.8, 25.6 (3C), 21.4 (d, J_{C-P}=6.4 Hz, 2C), 19.6 (d, J_{C-P}=1C), 17.7, −4.7, −4.8 δ ppm. ³¹P (202 MHz, DMSO-d₆): 3.49 δ ppm. HRMS (ESI) m/z: calcd for C₂₇H₄₀N₃O₉PSi [M-H]⁻: 608.2199; found 608.2178.

### f. Isopropyl ((S)-(((4R,5R)-5-(2,4-dioxo-3,4-dihydropyrimidin-1(2H)-yl)-4-hydroxy-4,5-dihydrofuran-2-yl)methoxy)(phenoxy)phosphoryl)-L-alaninate (HLB-0532255)

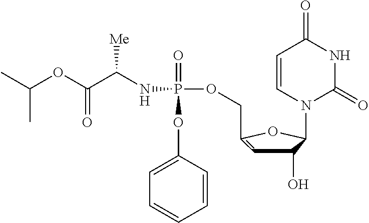 [SMILES: CC(C)OC(=O)[C@H](C)N[P@](=O)(OCC1=C[C@H](O)[C@H](n2ccc(=O)[nH]c2=O)O1)Oc1ccccc1]

Isopropyl ((S)-(((4R,5R)-4-((tert-butyldimethylsilyl)oxy)-5-(2,4-dioxo-3,4-dihydropyrimidin-1(2H)-yl)-4,5-dihydrofuran-2-yl)methoxy)(phenoxy)phosphoryl)-L-alaninate (230 mg, 1.0 mmol) was placed in a RB-flask equipped with a stir bar and dried under vacuum for 1 hr (placed on the high-vacuum). After drying for 1 hr, the RB-flask was purged with nitrogen and anhydrous THE was added (3 mL). The reaction was then cooled to 0° C. using an ice bath. To the reaction was added a 1:1 mixture of TBAF:AcOH (1.7 mmol). The mixture was stirred overnight under nitrogen at 0° C. After 24 hours, the reaction was concentrated in vacuo. The crude material was purified via column chromatography using 0-25% CH₃OH:CH₂Cl₂ over 16 minutes. The desired product eluted in 10% CH₃OH in CH₂Cl₂ as a white foam after concentrating in vacuo (116 mg, 62%). ¹H NMR (500 MHz, CDCl₃): 7.32 (t, J=7.9 Hz, 2H), 7.27-7.19 (m, 2H), 7.16 (t, J=7.2, 6.5 Hz, 2H), 6.29 (d, J=2.1 Hz, 1H), 5.69 (d, J=8.1 Hz, 1H), 5.34 (d, J=2.5 Hz, 1H), 5.01 (m, 1H), 4.89 (d, J=2.5 Hz, 1H), 4.76-4.64 (m, 2H), 4.11 (m, 1H), 3.94 (m, 1H), 1.36 (d, J=7.0 Hz, 3H), 1.23 (dd, J=6.3, 3.1 Hz, 6H) δ ppm. ¹³C NMR (126 MHz, CDCl₃): 173.1 (d, J_{C-P}=7.2 Hz, 1C), 163.3, 157.1 (d, J_{C-P}=7.0 Hz, 1C), 150.7 (d, J_{C-P}=6.6 Hz, 1C), 150.6, 139.1, 129.9 (3C), 125.3, 120.1 (d, J_{C-P}=4.9 Hz, 2C), 103.4 (d, J_{C-P}=5.8 Hz, 1C), 93.7, 79.6, 69.6, 60.7 (d, J_{C-P}=4.7 Hz, 1C), 50.4, 21.8 (d, J_{C-P}=12.1 Hz, 2C), 20.9 (d, J_{C-P}=5.2 Hz, 1C) δ ppm. ³¹P (202 MHz, CDCl₃): 2.54 δ ppm. HRMS (ESI) m/z: calcd for C₂₁H₂₆N₃O₉P [M-H]⁻: 494.1334; found 494.1323.

### Example 2. Synthesis of Isopropyl ((S)-(((4R,5R)-4-hydroxy-5-(5-methyl-2,4-dioxo-3,4-dihydropyrimidin-1(2H)-yl)-4,5-dihydrofuran-2-yl)methoxy)(phenoxy)phosphoryl)-L-alaninate

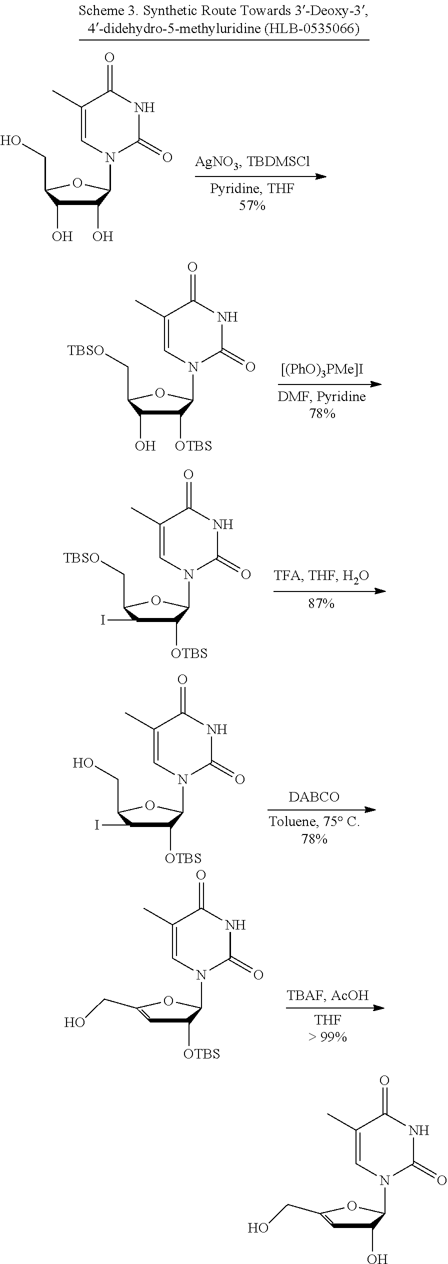

### a. 2′,5′-TBS-5-methyluridine

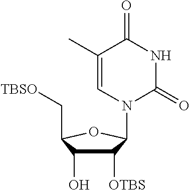

5-Methyluridine (Carbosynth, NM04922; 1.21 g, 1.0 mmol) was suspended in anhydrous THF (36 mL) followed by the addition of anhydrous pyridine (1.89 mL, 5 mmol) and silver nitrate (1.75 g, 2.2 mmol). The mixture stirred for 10 minutes before the addition of TBDMSCl (1.55 g, 2.2 mmol). The reaction stirred under nitrogen for 48 hours. After 48 hours, the reaction was quenched by the addition of ethanol (4 mL), filtered through a bed of celite, rinsing the celite with ethanol (2×25 mL) and the collected filtrate was concentrated in vacuo. The crude material was resuspended in ethyl acetate (25 mL), washed with water (3×30 mL), brine (1×30 mL), and the collected organics were dried over anhydrous Na₂SO₄, filtered, and concentrated in vacuo. The residue was purified by column chromatography using 0-30% EtOAc:Hex as eluent to give a white foam (1.31 g, 57%). HRMS (ESEI) m/z calcd for C₂₂H₄₂N₂O₆Si₂ [M-H]⁻: 485.2509; found 485.2499. This compound has been previously synthesized and characterization data agrees with previous reports (see Calvo-Mateo, A., et al., Tetrahedron 1988, 44 (15), 4895-4903; and Allan, A. L, et al., J Med Chem 2006, 49 (26), 7807-7815).

### b. 2′,5′-TBS-3′-iodo-5-methyluridine

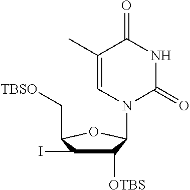

2′,5′-TBS-5-methyluridine (1.31 g, 1.0 mmol) was placed in a Schlenk flask and put on the high-vacuum for one hour before the addition of methyltriphenoxyphosphonium iodide (1.23 g, 1.01 mmol). The flask was then purged and filled with nitrogen 3 times. To this was added DMF (5 mL) and then pyridine (429 μL, 1.98 mmol). The reaction was stirred under nitrogen overnight wrapped in aluminum foil to protect from light. The reaction was quenched by the addition of CH₃OH (1 mL) and TEA (1 mL) and stirred for 15 minutes. The aqueous layer was extracted using ethyl acetate (20 mL) and washed with water 2× (20 mL) and brine 1× (20 mL). The collected organic layer was dried over sodium sulfate and concentrated in vacuo. The crude material was purified via flash column chromatography using hexanes:EtOAc from 0-30% over 20 minutes and a final push to 100% EtOAc for 5 minutes. The desired material was obtained as a yellow foam (1.26 g, 78%). ¹H NMR (500 MHz, DMSO-d₆): 11.38 (s, 1H), 7.38 (d, J=1.4 Hz, 1H), 5.62 (d, J=5.8 Hz, 1H), 4.42 (t, J=7.4 Hz, 1H), 4.39-4.33 (m, 1H), 4.04-3.97 (m, 1H), 3.81 (d, J=3.3 Hz, 2H), 1.73-1.66 (m, 3H), 0.83 (s, 9H), 0.72 (s, 9H), 0.04 (d, J=7.4 Hz, 6H), 0.00 (s, 3H), −0.17 (s, 3H) δ ppm. ¹³C NMR (126 MHz, DMSO-d₆): 163.3, 150.5, 134.7, 109.9, 86.5, 79.5, 77.9, 67.1, 26.3, 26.0 (3C), 25.4 (3C), 18.0, 17.5, 12.0, −4.2, −5.0, −5.2, −5.3 δ ppm. HRMS (ESI) m/z calcd for C₂₂H₄₁IN₂O₅Si₂ [M-H]⁻: 595.1526; found 595.1516.

### c. 3′-iodo-2′-TBS-5-methyluridine

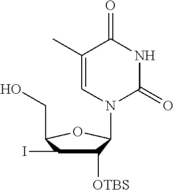

2′,5′-TBS-3′-iodo-5-methyluridine (1.26 g, 1.00 mmol) was dissolved in THE at 0° C. and to the mixture was added a 1:1 solution of TFA:H₂O (804 μL) dropwise over 3 minutes. The reaction mixture was warmed to RT and stirred for 24 hours. The reaction was neutralized by the addition of sat. aq NaHCO₃ and then extracted with EtOAc (3×25 mL). The combined organic layers were washed brine (1×30 mL) and dried over anhydrous Na₂SO₄, filtered, and concentrated in vacuo. The crude material was purified via flash chromatography from 0-100% EtOAc:hexanes. The desired material was obtained as a yellow foam (890 mg, 87%). ¹H NMR (500 MHz, DMSO-d₆): 11.41 (bs, 1H), 7.79 (d, J=1.4 Hz, 1H), 5.68 (d, J=5.1 Hz, 1H), 4.60 (dd, J=6.4, 5.1 Hz, 1H), 4.46 (t, J=6.6 Hz, 1H), 4.03-3.97 (m, 1H), 3.80-3.67 (m, 2H), 1.79 (s, 3H), 0.84 (s, 9H), 0.11 (s, 3H), −0.04 (s, 3H) δ ppm. ¹³C NMR (126 MHz, DMSO-d₆): 163.2, 150.3, 135.6, 109.3, 87.5, 80.6, 78.7, 65.1, 27.7, 25.1 (3C), 17.2, 11.9, −4.6, −5.4 δ ppm. HRMS (ESI) m/z: calcd for C₁₆H₂₇IN₂O₅Si [M-H]⁻: 481.0661; found 481.0647.

### d. 3′-Deoxy-3′,4′-didehydro-2′-TBS-5-methyluridine

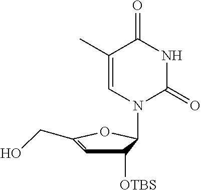 [SMILES: Cc1cn([C@@H]2OC(CO)=C[C@@H]2O[Si](C)(C)C(C)(C)C)c(=O)[nH]c1=O]

3′-iodo-2′-TBS-5-methyluridine (890 mg, 1 mmol) was dissolved in toluene (30 mL) and to the solution was added DABCO (724 mg, 3.5 mmol). The reaction was heated to 75° C. and stirred overnight. The next day, the reaction mixture was cooled and filtered to remove salts formed. The collected filtrate was concentrated in vacuo and purified via flash chromatography using EtOAc:hexanes from 40-100%. The desired material collected was obtained as a yellow foam (500 mg, 76%). ¹H NMR (500 MHz, DMSO-d₆): 11.46 (s, 1H), 7.12 (d, J=1.3 Hz, 1H), 6.13 (d, J=2.5 Hz, 1H), 5.31 (t, J=5.9 Hz, 1H), 5.18-5.13 (m, 1H), 5.05-5.00 (m, 1H), 4.05 (dt, J=5.9 Hz, 2H), 1.76 (d, J=1.2 Hz, 3H), 0.85 (s, 9H), 0.05 (d, J=5.0 Hz, 6H) δ ppm. 13C NMR (126 MHz, DMSO-d₆): 163.6, 162.3, 150.1, 135.1, 110.4, 99.9, 92.2, 79.4, 56.0, 25.6 (3C), 17.7, 12.1, −4.6, −4.8 δ ppm. HRMS (ESI) m/z: calcd for C₁₆H₂₆N₂O₅Si [M-H]⁻: 353.1538; found 353.1529.

### e. 3′-Deoxy-3′,4′-didehydro-5-methyluridine

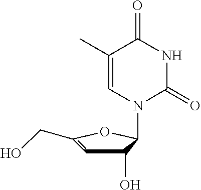 [SMILES: Cc1cn([C@@H]2OC(CO)=C[C@@H]2O)c(=O)[nH]c1=O]

3′-Deoxy-3′,4′-didehydro-2′-TBS-5-methyluridine (436 mg, 1.0 mmol) was dissolved in THF (15 mL). To this was added a 1:1 mixture of acetic acid:TBAF (1M in THF; 1.48 mmol). The reaction stirred overnight. The next day, the reaction was concentrated in vacuo and purified via flash chromatography using CH₃OH:CH₂Cl₂ from 0-25% over 20 minutes. 3′-Deoxy-3′,4′-didehydro-5-methyluridine eluted in 10% CH₃OH in CH₂Cl₂ and formed a white foam after concentrating in vacuo (400 mg, >99%). ¹H NMR (500 MHz, DMSO): 7.05 (d, J=1.4 Hz, 1H), 6.09 (d, J=2.6 Hz, 1H), 5.57 (s, 1H), 5.12-5.07 (m, 1.2 Hz, 1H), 4.79-4.75 (m, 1H), 3.99 (s, 2H), 2.48-2.44 (m, 1H), 1.72 (d, J=1.3 Hz, 3H) δ ppm. ¹³C NMR (151 MHz, DMSO): 163.7, 161.6, 150.3, 135.3, 110.5, 100.2, 92.2, 77.5, 56.2, 12.1 δ ppm. HRMS (ESI) m/z: calcd for C₁₀H₁₂N₂O₅ [M-H]239.0673; found 239.0663.

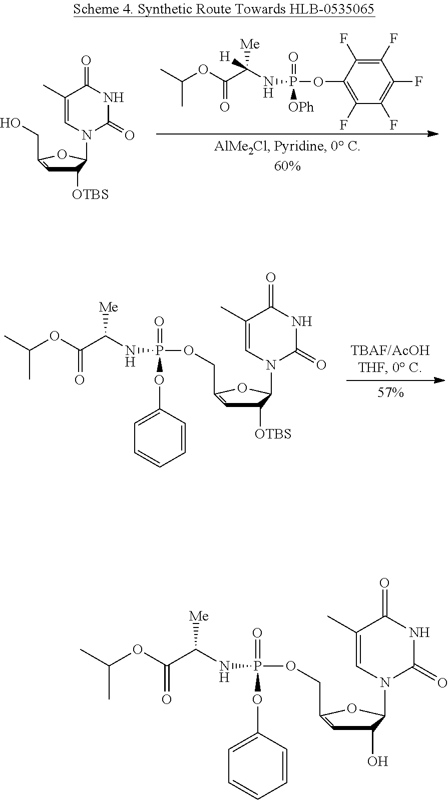 [SMILES: Cc1cn([C@@H]2OC(CO)=C[C@@H]2O[Si](C)(C)C(C)(C)C)c(=O)[nH]c1=O ; CC(C)OC(=O)[C@H](C)N[P@@](=O)(Oc1ccccc1)Oc1c(F)c(F)c(F)c(F)c1F ; Cc1cn([C@@H]2OC(CO[P@@](=O)(N[C@@H](C)C(=O)OC(C)C)Oc3ccccc3)=C[C@@H]2O[Si](C)(C)C(C)(C)C)c(=O)[nH]c1=O ; Cc1cn([C@@H]2OC(CO[P@@](=O)(N[C@@H](C)C(=O)OC(C)C)Oc3ccccc3)=C[C@@H]2O)c(=O)[nH]c1=O]

### f. Isopropyl ((S)-(((4R,5R)-4-((tert-butyldimethylsilyl)oxy)-5-(5-methyl-2,4-dioxo-3,4-dihydropyrimidin-1(2H)-yl)-4,5-dihydrofuran-2-yl)methoxy)(phenoxy)phosphoryl)-L-alaninate

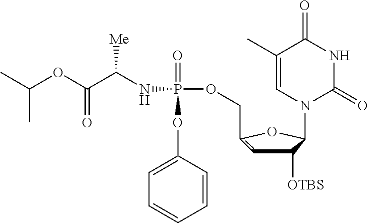 [SMILES: Cc1cn([C@@H]2OC(CO[P@@](=O)(N[C@@H](C)C(=O)OC(C)C)Oc3ccccc3)=C[C@@H]2O[Si](C)(C)C(C)(C)C)c(=O)[nH]c1=O]

3′-Deoxy-3′,4′-didehydro-2′-TBS-5-methyluridine (104 mg, 1.0 mmol) and propan-2-yl (2S)-2-{[(2,3,4,5,6 pentafluorophenoxy)(phenoxy)phosphoryl]amino}propanoate (199 mg, 1.5 mmol) were placed in a Schlenk flask equipped with a stir bar and was placed under vacuum using a needle and septa to dry overnight (placed on the high-vacuum). The next morning, the Schlenk flask was purged with nitrogen and anhydrous pyridine was added (3 mL). The reaction was then cooled to 0° C. using an ice bath and stirred for 20 minutes before the dropwise addition of dimethylaluminum chloride (AlMe₂Cl, 1M in hexanes, 147 μL, 0.5 mmol). The reaction was stirred at 0° C. for 30 minutes before removing the ice bath and letting the reaction warm-up to RT. The reaction was stirred at RT for 48 hours. The reaction was quenched by 30% w/v aq. tartaric acid (1 mL) at 0° C. for 1 minute. The reaction was then returned to RT and mixed for 5 minutes. The solution was then extracted with EtOAc (5 mL, ×3) and washed with brine (10 mL, ×2). The collected organic layers were then dried over Na₂SO₄, filtered, and the filtrate was concentrated in vacuo. The crude material was purified via column chromatography using 0-25% CH₃OH:CH₂Cl₂ over 16 minutes. The desired material was obtained as a white foam (110 mg, 60%). ¹H NMR (500 MHz, DMSO-d₆): 11.48 (s, 1H), 7.39-7.32 (m, 2H), 7.26-7.13 (m, 4H), 6.19-6.15 (m, 1H), 6.09 (dd, J=13.2, 10.0 Hz, 1H), 5.38-5.31 (m, 1H), 5.12-5.06 (m, 1H), 4.91-4.80 (m, 1H), 4.71-4.63 (m, 2H), 3.84-3.72 (m, 1H), 1.77-1.68 (m, 3H), 1.26-1.19 (m, 3H), 1.19-1.13 (m, 6H), 0.88-0.81 (s, 9H), 0.09-0.04 (d, J=3.3 Hz, 6H) δ ppm. ¹³C NMR (126 MHz, DMSO-d₆): 172.5 (d, J_{C-P}=5.0 Hz, 1C), 163.6, 156.4 (d, J_{C-P}=8.3 Hz, 1C), 150.6 (d, J_{C-P}=6.4 Hz, 1C), 150.1, 135.3, 129.5 (2C), 124.6, 120.1 (d, J_{C-P}=4.5 Hz, 2C), 110.6, 103.2, 92.5, 79.3, 67.9, 59.9 (d, J_{C-P}=4.5 Hz, 1C), 49.8, 25.6 (3C), 21.3 (d, J_{C-P}=5.5 Hz, 2C), 19.6 (d, J_{C-P}=6.5 Hz, 1C), 17.7, 12.0, −4.7, −4.8 δ ppm. ³¹P (202 MHz, DMSO-d₆): 3.48 δ ppm. HRMS (ESI) m/z: calc'd for C₂₈H₄₁N₃O₉PSi 622.2355 [M-H]⁻; found 622.2275.

### g. Isopropyl ((S)-(((4R,5R)-4-hydroxy-5-(5-methyl-2,4-dioxo-3,4-dihydropyrimidin-1(2H)-yl)-4,5-dihydrofuran-2-yl)methoxy)(phenoxy)phosphoryl)-L-alaninate

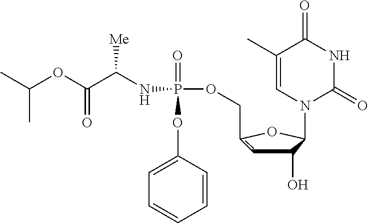 [SMILES: Cc1cn([C@@H]2OC(CO[P@@](=O)(N[C@@H](C)C(=O)OC(C)C)Oc3ccccc3)=C[C@@H]2O)c(=O)[nH]c1=O]

Isopropyl ((S)-(((4R,5R)-4-((tert-butyldimethylsilyl)oxy)-5-(5-methyl-2,4-dioxo-3,4-dihydropyrimidin-1(2H)-yl)-4,5-dihydrofuran-2-yl)methoxy)(phenoxy)phosphoryl)-L-alaninate (110 mg, 1.0 mmol) was placed in a RB-flask equipped with a stir bar and was placed under vacuum for 1 hr (placed on the high-vacuum). After drying for 1 hr, the RB flask was purged with nitrogen and anhydrous THE was added (3 mL). The reaction was then cooled to 0° C. using an ice bath. To the reaction was added a mixture of 1 M TBAF in THF/AcOH (1:1 mixture, 1.7 mmol). The flask was stirred overnight under nitrogen at 0° C. The reaction was concentrated in vacuo. The crude material was purified via column chromatography using 0-25% CH₃OH:CH₂Cl₂ over 16 minutes. The desired material was obtained as a white foam (50.7 mg, 57%). ¹H NMR (500 MHz, DMSO-d₆): 12.24 (s, 1H), 8.16-8.09 (m, 2H), 8.01-7.89 (m, 4H), 6.93 (d, J=2.9 Hz, 1H), 6.84 (dd, J=13.2, 10.0 Hz, 1H), 6.51 (d, J=6.1 Hz, 1H), 6.10 (d, J=2.4 Hz, 1H), 5.67-5.58 (m, 2H), 5.40 (d, J=7.8 Hz, 1H), 4.61-4.49 (m, 1H), 4.09 (s, 1H), 3.27 (p, J=1.8 Hz, 3H), 1.99 (d, J=7.1 Hz, 3H), 1.92 (d, J=6.3 Hz, 6H) δ ppm. ¹³C (151 MHz, DMSO): 172.7 (d, J_{C-P}=5.3 Hz, 1C), 163.7, 155.9, 155.8, 150.8 (d, J_{C-P}=6.5 Hz, 1C), 150.3, 135.6, 129.7, 124.7, 120.2 (d, J_{C-P}=4.7 Hz, 1C), 110.7, 104.0, 92.6, 77.5, 68.1, 60.2, 60.1, 49.9, 21.5, 21.3, 19.8 (d, J_{C-P}=6.5 Hz, 1C), 12.1 δ ppm. ³¹P (202 MHz, DMSO): 3.50 δ ppm. HRMS (ESI) m/z: calcd for C₂₂H₂₈N₃O₉P [M-H]⁻: 508.1490; found 508.1480.

### Example 3. Synthesis of Isopropyl ((S)-(((4R,5R)-4-hydroxy-5-(6-oxo-1,6-dihydro-9H-purin-9-yl)-4,5-dihydrofuran-2-yl)methoxy)(phenoxy)phosphoryl)-L-alaninate

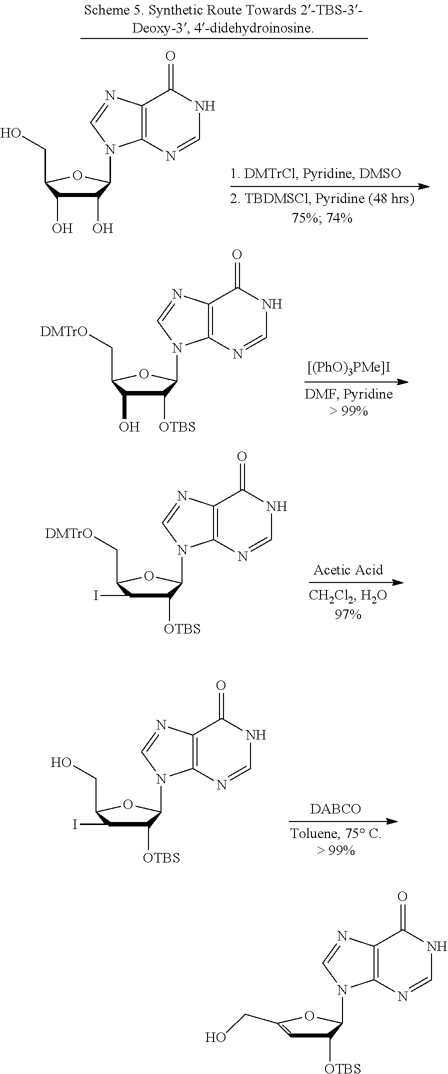

### a. 5D T-TBS-inosine

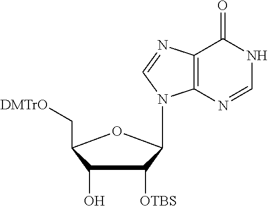

In a Schlenk flask was added inosine (Carbosynth, NI06297; 1.00 g, 1.00 mmol) and the material was dried overnight under high-vacuum at 100° C. The next day, DMTrCl (2.35 g, 1.2 mmol) was added in one portion to a suspension of inosine in anhydrous pyridine and DMSO as a 1:1 solution (10 mL total). The reaction mixture was stirred at RT overnight. The next day, the reaction was quenched by the addition of methanol (2 mL) and stirred for 15 minutes. The solution was partitioned between CH₂Cl₂ (30 mL) and sat. aq. NaHCO₃ (30 mL). The aqueous layer was extracted with CH₂Cl₂, the organic layers were combined, washed with brine (30 mL) and dried over Na₂SO₄. The organic solvent was removed in vacuo and the residue was co-evaporated twice with toluene to remove residual pyridine and DMSO. Flash silica gel column chromatography was used to purify the material using CH₃OH:CH₂Cl₂ from 0-25% CH₃OH:CH₂Cl₂, removing the bis-substituted products and unreacted starting material. The 2′-TBS-inosine was obtained as a white foam (1.68 g, 79%).

To 2′-TBS-inosine was added anhydrous THE (20 mL) and pyridine (2 mL) under argon. AgNO₃ (650 mg, 1.3 mmol) was added and stirred until the mixture fully dissolved. TBDMSCl (577 mg, 1.3 mmol) was then added to the mixture and stirred overnight at RT. The next day an additional 0.3 eq of AgNO₃ (150 mg) and 0.3 eq of TBDMSCl (133 mg) was added and stirred for another 24 hours at RT. The mixture was then filtered through a pad of celite into saturated aqueous NaHCO₃ solution and the filtrate extracted with CH₂Cl₂ (3×30 mL). The organic layers were combined, washed with brine (1×30 mL), dried over Na₂SO₄ and concentrated in vacuo. The residue was dissolved in refluxing EtOAc, the white precipitate which forms on standing at RT was filtered and dried. The mother liquor which contains mono and bis substituted derivatives were then concentrated in vacuo and then dissolved in 3% TEA in methanol. After 3 hours at RT the solvents were concentrated in vacuo and the residue was crystallized as above to afford more of the desired material (1.49 g, 74%). HRMS (ESI) m/z: calcd for C₃₇H₄₄N₄O₇Si [M-H]⁻: 683.2906; found 683.2884. This compound has been previously synthesized and characterization data agrees with previous reports (See Matulic-Adamic, J. and Beigelman, L. Synth Commun 2000, 30 (21), 3963-3969.

### b. 5′-DMT-3′-iodo-2′-TBS-inosine

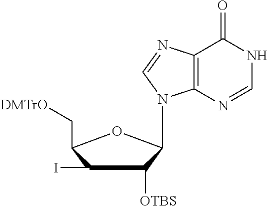

5′-DMT-2′-TBS-inosine (721 mg, 1.00 mmol) was placed in a Schlenk flask and placed on the high-vacuum for one hour before the addition of methyltriphenoxyphosphonium iodide (481 mg, 1.01 mmol). The flask was then purged and filled with nitrogen 3 times. To this was added DMF (5 mL) and then pyridine (168 μL, 1.98 mmol). The reaction was stirred under nitrogen overnight wrapped in aluminum foil to protect from light. The reaction was quenched by the addition of CH₃OH (1 mL) and TEA (1 mL) and stirred for 15 minutes. The aqueous layer was extracted using ethyl acetate (20 mL) and washed with water 2× (20 mL) and brine 1× (20 mL). The organic layer was dried over sodium sulfate and concentrated in vacuo. The crude material was purified via flash column chromatography using hexanes:EtOAc from 0-30% over 20 minutes and a final push to 100% EtOAc for 5 minutes. 5′-DMT-3′-iodo-2′-TBS-inosine was obtained as a yellow foam (1.42 g, >99%). HRMS (ESI) m/z: calcd for C₃₇H₄₃IN₄O₆Si [M-H]⁻: 793.1924; found 793.1898.

### c. 3′-iodo-2′-TBS-inosine

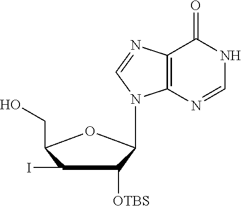

5′-DMT-3′-iodo-2′-TBS-inosine (700 mg, 1.00 mmol) was dissolved in CH₂Cl₂ (10 mL) and to this solution was added a 4:1 mixture of acetic acid:water (10 mL:2.5 mL). The reaction mixture was stirred at RT overnight. The reaction was then adjusted to a pH of 7 by the addition of saturated aqueous NaHCO₃ and the reaction was extracted in EtOAc (30 mL). The organic layer was washed with water (2×30 mL) and brine (1×30 mL). The collected organic layer was then dried over Na₂SO₄, filtered and concentrated in vacuo. The crude compound was purified on silica gel using hexanes:EtOAc up to 100% EtOAc. 3′-iodo-2′-TBS-inosine was obtained as a yellow foam (420 mg, 97%). ¹H NMR (601 MHz, DMSO-d₆): 11.99 (s, 1H), 8.48 (s, 1H), 6.53 (d, J=6.3 Hz, 1H), 5.94-5.90 (m, 1H), 5.00 (dd, J=6.4, 4.5 Hz, 1H), 4.86 (dd, J=4.5, 2.5 Hz, 1H), 4.61-4.57 (m, 1H), 4.42-4.36 (m, 1H), 4.32-4.26 (m, 1H), 3.98 (s, 1H), 1.79 (s, 1H), 1.54 (s, 9H), 0.70 (d, J=38.3 Hz, 6H) δ ppm. ¹³C (151 MHz, DMSO-d₆): 164.0, 151.2, 136.5, 110.0, 87.0, 86.0, 74.7, 72.5, 61.0, 26.2 (3C), 18.2, 12.5, −4.2, −4.3 δ ppm. HRMS (ESI) m/z: calcd for C₁₆H₂₅IN₄O₄Si [M-H]⁻: 491.0617; found 491.0604.

### d. 3′-Deoxy-3′,4′-didehydro-2′-TBS-inosine

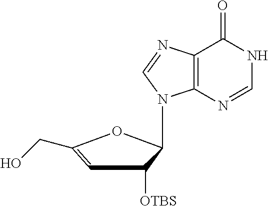

3′-iodo-2′-TBS-inosine (420 mg, 1.00 mmol) was dissolved in toluene (15 mL) and to the solution was added DABCO (335 mg, 3.5 mmol). The reaction was mixed at 75° C. overnight. The next day, the reaction mixture was cooled and filtered to remove salts formed. The collected filtrate was concentrated in vacuo and purified via flash chromatography using hexanes:EtOAc from 40-100%. The desired material was obtained as a yellow foam (400 mg, >99%). ¹H NMR (601 MHz, DMSO-d₆): 12.39 (s, 1H), 8.14 (s, 1H), 8.10 (s, 1H), 6.26 (d, J=2.6 Hz, 1H), 5.41-5.37 (m, 1H), 5.27-5.23 (m, 2H), 4.06-4.03 (m, 2H), 0.86-0.80 (m, 9H), 0.02 (d, J=5.0 Hz, 6H) δ ppm. ¹³C (151 MHz, DMSO-d₆): 162.2, 156.9, 148.3, 146.8, 138.2, 124.7, 100.1, 92.0, 80.0, 56.6, 26.1 (3C), 18.1, −4.1, −4.3 δ ppm. HRMS (ESI) m/z: calcd for C₁₆H₂₄N₄O₄Si [M-H]⁻: 363.1494; found 363.1477.

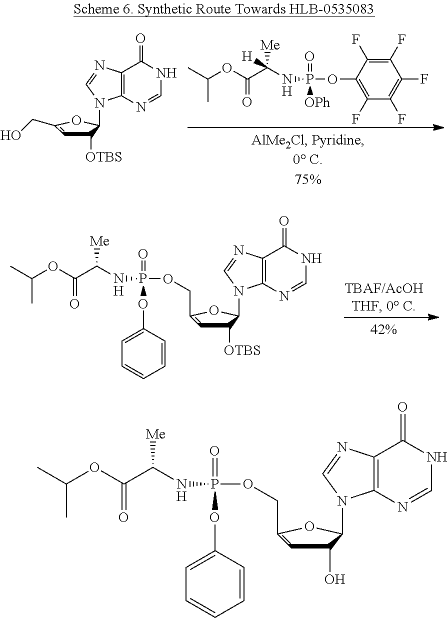

### e. Isopropyl ((S)-(((4R,5R)-4-((tert-butyldimethylsilyl)oxy)-5-(6-oxo-1,6-dihydro-9H-purin-9-yl)-4,5-dihydrofuran-2-yl)methoxy)(phenoxy)phosphoryl)-L-alaninate

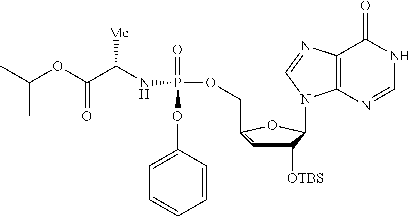 [SMILES: CC(C)OC(=O)[C@H](C)N[P@](=O)(OCC1=C[C@H](O[Si](C)(C)C(C)(C)C)[C@H](n2cnc3c(=O)[nH]cnc32)O1)Oc1ccccc1]

3′-Deoxy-3′,4′-didehydro-2′-TBS-inosine (254 mg, 1.00 mmol) and propan-2-yl (2S)-2-{[(2,3,4,5,6 pentafluorophenoxy)(phenoxy)phosphoryl]amino}propanoate (316 mg, 1.5 eq.) were placed in a Schlenk flask equipped with a stir bar and was placed under vacuum using a needle and septa (placed on the high-vacuum). The next morning, the Schlenk flask was purged with nitrogen and anhydrous pyridine was added (10 mL). The reaction was then cooled to 0° C. using an ice bath and stirred for 20 minutes before the dropwise addition of dimethyl aluminum chloride (AlMe₂C, 1M in hexanes). The reaction was stirred at 0° C. for 30 minutes before removing the ice bath and letting the reaction warm-up to RT. The reaction was stirred at RT for 48 hours. After 48 hours, the reaction was quenched by 30% w/v aq. tartaric acid (1 mL) at 0° C. for 1 minute. The reaction was then returned to RT and mixed for 5 minutes. The solution was then extracted with EtOAc (5 mL, ×3) and washed with brine (10 mL, ×2). The collected organic layers were then dried over Na₂SO₄, filtered, and the filtrate was concentrated in vacuo. The crude material was purified via column chromatography using 0-25% CH₃OH:CH₂Cl₂ over 16 minutes. The desired material was obtained as a white foam (330 mg, 75%).

### f. Isopropyl ((S)-(((4R,5R)-4-hydroxy-5-(6-oxo-1,6-dihydro-9H-purin-9-yl)-4,5-dihydrofuran-2-yl)methoxy)(phenoxy)phosphoryl)-L-alaninate

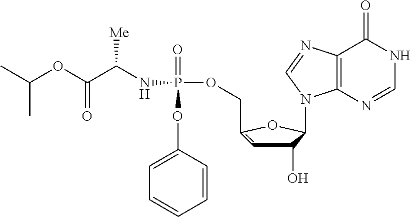 [SMILES: CC(C)OC(=O)[C@H](C)N[P@](=O)(OCC1=C[C@H](O)[C@H](n2cnc3c(=O)[nH]cnc32)O1)Oc1ccccc1]

Isopropyl ((S)-(((4R,5R)-4-((tert-butyldimethylsilyl)oxy)-5-(6-oxo-1,6-dihydro-9H-purin-9-yl)-4,5-dihydrofuran-2-yl)methoxy)(phenoxy)phosphoryl)-L-alaninate (330 mg, 1.00 mmol) was placed in a RB-flask equipped with a stir bar and was placed under vacuum for 1 hr. After drying for 1 hr, the RB flask was purged with nitrogen and anhydrous THE was added (3 mL). The reaction was then cooled to 0° C. using an ice bath. To the reaction was added a mixture of 1M TBAF in THF:AcOH (1:1 mixture, 1.7 mmol). The mixture was stirred overnight under nitrogen at 0° C. The next day, the solvent was concentrated in vacuo. The crude material was purified via column chromatography using 0-25% CH₃OH:CH₂Cl₂ over 16 minutes. The desired material was obtained as a white foam (115 mg, 42%). ¹H NMR (500 MHz, DMSO-d₆): 8.14 (s, 1H), 8.10 (s, 1H), 7.36-7.28 (m, 2H), 7.21-7.12 (m, 3H), 6.27 (d, J=2.8 Hz, 1H), 6.04 (dd, J=13.2, 10.0 Hz, 1H), 5.90 (s, 1H), 5.44 (d, J=2.5 Hz, 1H), 5.23 (s, 1H), 4.86-4.81 (m, 1H), 4.67-4.58 (m, 2H), 3.83-3.71 (m, 1H), 1.21 (d, J=7.1 Hz, 3H), 1.12 (t, J=5.9 Hz, 6H) δ ppm. 13C NMR (151 NMR, DMSO-d₆): 172.5 (d, J_{C-P}=5.1 Hz, 1C), 156.5, 155.0, 155.0, 150.6 (d, J_{C-P}=6.4 Hz, 1C), 147.8, 146.3, 138.0, 129.5, 124.5, 124.2, 120.1 (d, J_{C-P}=4.8 Hz, 1C), 103.7, 91.6, 77.5, 67.9, 59.9, 59.9, 49.7, 21.3, 21.3, 19.67 (d, J_{C-P}=6.7 Hz, 1C) δ ppm. ³¹p NMR (202 NMR, DMSO): 3.53 δ ppm. HRMS (ESI) m/z: calcd for C₂₂H₂₆N₅O₈P [M-H]⁻: 518.1446; found 518.1436.

### Example 4. Synthesis of Isopropyl ((S)-(((4R,5R)-5-(2-amino-6-oxo-1,6-dihydro-9H-purin-9-yl)-4-hydroxy-4,5-dihydrofuran-2-yl)methoxy)(phenoxy)phosphoryl)-L-alaninate

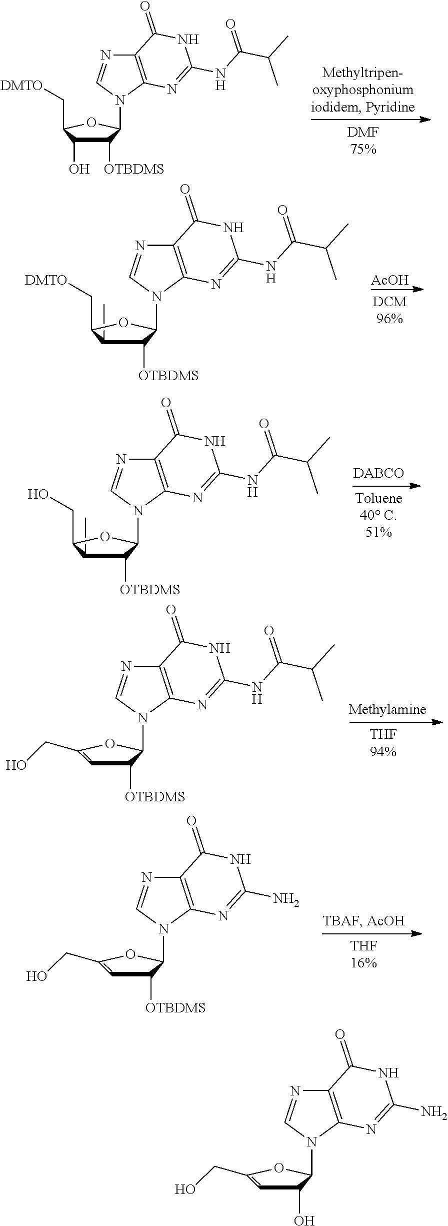

### a. 5′-DMT-3′-Iodo-2′-TBDMS-N2-isobutyryl-guanosine

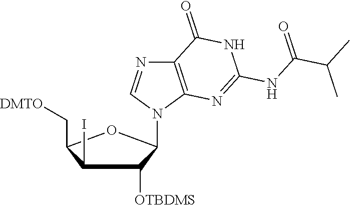

N-[9-(5-{[bis(4-methoxyphenyl)(phenyl)methoxy]methyl}-3-[(tert-butyldimethylsilyl)oxy]-4-hydroxyoxolan-2-yl)-6-oxo-6,9-dihydro-1H-purin-2-yl]-2-methylpropanamide (1.54 g, 2.00 mmol) was placed in a Schlenk flask and placed on the high-vacuum for one hour before the addition of methyltriphenoxyphosphonium iodide (1.45 g, 3.20 mmol). The flask was then purged and filled with nitrogen 3 times before the addition of solvent. To the purged flask was added DMF (13.2 mL) and then pyridine (320 μL, 2.63 mmol). The reaction was stirred under nitrogen overnight (18 hours). After 18 hours, the reaction was quenched by the addition of CH₃OH (1.0 mL) and TEA (1.0 mL) and stirred for 15 minutes. The reaction mixture was diluted with ethyl acetate (20 mL), washed with water two times (20 mL), brine (20 mL), filtered through sodium sulfate and concentrated in vacuo. The residue was purified by chromatography on silica gel with a gradient of 0-30% EtOAc in hexanes to yield 5′-DMT-3′-Iodo-2′-TBDMS-N2-isobutyryl-guanosine (1.32 g, 75% yield). ¹H NMR (500 MHz, MeOD): 8.03 (s, 1H), 7.46 (m, 2H), 7.34 (m, 4H), 7.26 (m, 3H), 6.84 (m, 4H), 5.87 (d, J=5.5 Hz, 1H), 5.08 (t, J=6.5 Hz, 1H), 4.48 (t, J=6.5 Hz, 1H), 4.19 (m, 1H), 3.77 (s, 6H), 3.61 (m, 2H), 2.66 (m, 1H), 1.21 (d, J=1.5 Hz, 3H), 1.19 (d, J=1.5 Hz, 3H), 0.83 (s, 9H), 0.18 (s, 3H), −0.20 (s, 3H) δ ppm. ¹³C NMR (126 MHz, CD₃OD): 181.7, 160.4, 160.4, 157.4, 150.9, 150.1, 145.8, 139.6, 136.6, 136.6, 131.7, 131.6, 129.7, 128.8, 128.2, 121.3, 114.1, 89.4, 88.4, 84.2, 80.4, 69.1, 55.7, 36.9, 27.9, 26.1, 19.4, 19.2, 18.7, −3.8, −4.8 δ ppm. HRMS (ESI) m/z: calcd for C₄₁H₅₀IN₅O₇Si [M-H]⁻: 878.2451; found 878.2421.

### b. 3′-Iodo-2′-TBDMS-N2-isobutyryl-guanosine

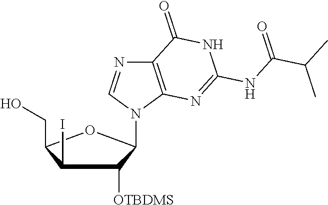

5′-DMT-3′-Iodo-2′-TBDMS-N2-isobutyryl-guanosine (1.36 g, 1.55 mmol) was dissolved in CH₂Cl₂ (1.02 mL) and to this solution was added a 4:1 mixture of acetic acid:water (2.08 mL:512 μL). The reaction mixture was stirred overnight. The reaction mixture was diluted with ethyl acetate (20 mL), washed with water two times (20 mL), brine (20 mL), filtered through sodium sulfate and concentrated in vacuo. The residue was purified by chromatography on silica gel with a gradient of 0-100% EtOAc in hexanes to yield 3′-Iodo-2′-TBDMS-N2-isobutyryl-guanosine (857 mg, 96% yield). ¹H NMR (500 MHz, CDCl₃): 12.26 (s, 1H), 7.99 (s, 1H), 5.53 (d, J=6.0 Hz, 1H), 5.00 (q, J=6.5 Hz, J=1.5 Hz, 1H), 4.40 (t, J=8.0 Hz, 1H), 4.31 (m, 1H), 4.09 (s, 2H), 2.80 (s, 1H), 1.26 (m, 6H), 0.80 (s, 9H), 0.08 (s, 3H), −0.38 (s, 3H) δ ppm. 13C NMR (126 MHz, CD₃CN): 181.0, 156.2, 149.7, 149.3, 139.1, 122.1, 89.1, 82.4, 80.8, 66.9, 36.6, 27.7, 25.9, 19.2, 19.1, 18.4, −3.9, −5.1 δ ppm. HRMS (ESI) m/z: calc'd for C₂₀H₃₁IN₅O₅Si [M-H]⁻: 576.1145; found 576.1068.

### c. 3′-Deoxy-3′,4′-didehydro-2′-TBDMS-N2-isobutyryl-guanosine

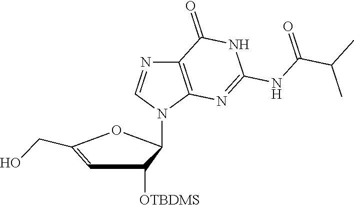

3′-Iodo-2′-TBDMS-N2-isobutyryl-guanosine (100 mg, 173 mol) was suspended in toluene (2.0 mL) and to the solution was added DABCO (68.0 mg, 606 mol). The reaction mixture was then heated to 40° C. and stirred overnight (18 hours). The reaction mixture was cooled and filtered to remove the salts formed. The collected filtrate was concentrated in vacuo. The residue was purified by chromatography on silica gel with a gradient of 0-10% CH₃OH in CH₂Cl₂ to yield 3′-Deoxy-3′,4′-didehydro-2′-TBDMS-N2-isobutyryl-guanosine (40.0 mg, 51% yield). ¹H NMR (500 MHz, CDCl₃): 7.98 (s, 1H), 6.31 (d, J=2.0 Hz, 1H), 5.33 (m, 1H), 5.32 (m, 1H), 4.17 (q, J=14.5 Hz, J=5.5 Hz, 2H), 2.73 (m, 1H), 1.23 (s, 3H), 1.22 (s, 3H), 0.89 (s, 9H), 0.08 (s, 3H), 0.07 (s, 3H) δ ppm. ¹³C NMR (126 MHz): 181.9, 163.2, 157.5, 150.4, 150.2, 138.5, 120.9, 101.2, 92.8, 81.7, 57.9, 36.9, 26.2, 19.4, 19.3, 18.9, −4.4, −4.5 δ ppm. HRMS (ESI) m z: calc'd for C₂₀H₃₀N₅O₅Si [M-H]⁻: 448.2022; found 448.1961.

### d, 3′-Deoxy-3′,4′-didehydro-2′-TBDMS-guanosine

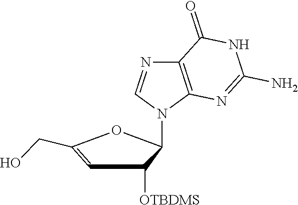

3′-Deoxy-3′,4′-didehydro-2′-TBDMS-N2-isobutyryl-guanosine (500 mg, 1.11 mmol) was dissolved in a solution of methylamine in THE (2.0 M solution, 11.3 mL). This was stirred overnight. The reaction was concentrated in vacuo causing the product to precipitate. The solid residue was filtered with EtOAc (20 mL) and dried in vacuo to yield 3′-Deoxy-3′,4′-didehydro-2′-TBDMS-guanosine (398 mg, 94% yield). ¹H NMR (500 MHz, DMSO-d₆): 10.68 (s, 1H), 7.72 (s, 1H), 6.53 (s, 2H), 6.04 (d, J=3.0 Hz, 1H), 5.35 (s, 1H), 5.26 (t, J=6.0, 1H), 5.20 (s, 1H), 4.00 (d, J=6.0 Hz, 2H), 0.83 (s, 9H), 0.03 (s, 3H), 0.01 (s, 3H) δ ppm. ¹³C (126 MHz, CD₃OD): 163.1, 159.3, 155.6, 152.9, 136.5, 117.4, 101.0, 92.8, 81.6, 58.0, 26.2, 18.9, −4.5, −4.5 δ ppm. HRMS (ESI) m/z: calc'd for C₁₆H₂₄N₅O₄Si [M-H]⁻: 378.1603; found 378.1550.

### e. 3′-Deoxy-3′,4′-didehydroguanosine

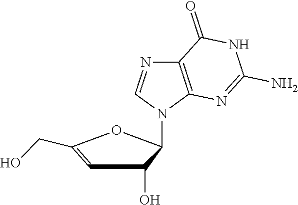

3′-Deoxy-3′,4′-didehydro-2′-TBDMS-guanosine (393 mg, 1.04 mmol) was dissolved in THE (7.67 mL). To this was added a 1:1 mixture of acetic acid:tetrabutylammonium fluoride (1.25 mL: 1.25 mL). The reaction was stirred overnight. The reaction was concentrated in vacuo. The residue was purified by chromatography on silica gel with a gradient of 0-10% CH₃OH in CH₂Cl₂ to yield 3′-Deoxy-3′,4′-didehydroguanosine (44 mg, 16% yield). ¹H NMR (500 MHz, DMSO-d₆): 10.66 (s, 1H), 7.63 (s, 1H), 6.54 (s, 2H), 6.04 (d, J=2.5 Hz, 1H), 5.69 (d, J=6.5 Hz, 1H), 5.23 (t, J=6.0 Hz, 1H), 5.20 (m, 1H), 5.06 (m, 1H), 4.00 (d, J=5.5 Hz, 2H) δ ppm. ¹³C (126 MHz, DMSO-d₆): 161.1, 156.7, 154.0, 151.0, 134.2, 116.2, 99.8, 90.4, 77.4, 56.3 δ ppm. HRMS (ESI) m/z: calcd for C₁₀H₁₁N₅O₄ [M-H]⁻: 264.0738; found 264.0732.

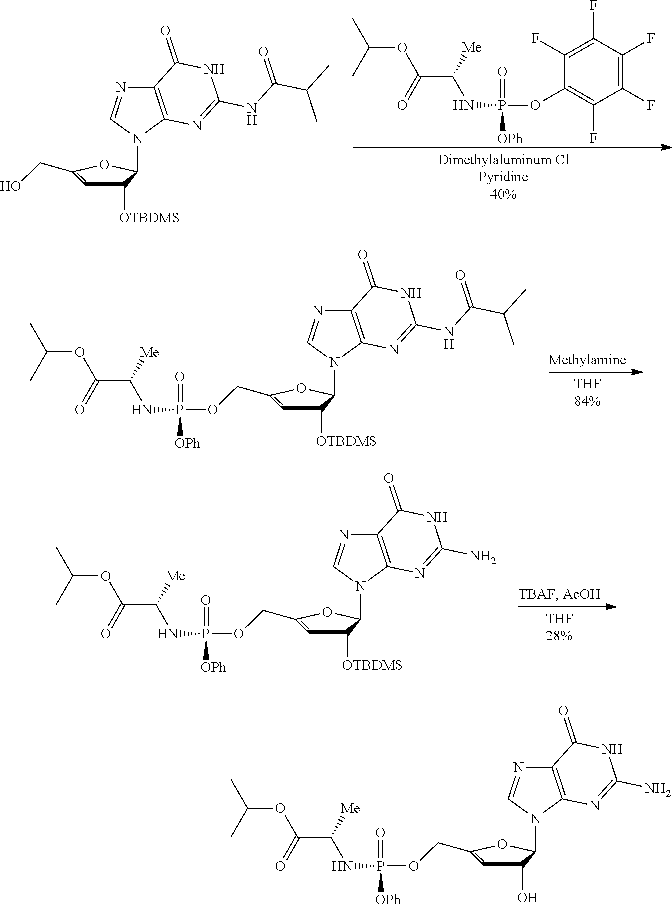

### f. Propan-2-yl (2S)-2-{[(S)-{[(4R,5R)-5-(2-amino-6-oxo-6,9-dihydro-1H-purin-9-yl)-4-hydroxy-4,5-dihydrofuran-2-yl]methoxy}(phenoxy)-phosphoryl]amino}propanoate

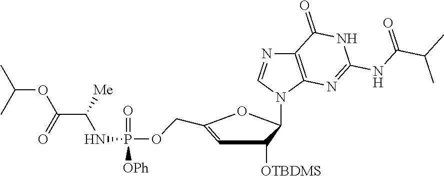

3′-Deoxy-3′,4′-didehydro-2′-TBDMS-N2-isobutyryl-guanosine (320 mg, 712 mol) and propan-2-yl (2S)-2-{[(2,3,4,5,6 pentafluoro-phenoxy)(phenoxy)phosphoryl]amino}-propanoate (484 mg, 1.07 mmol) were placed in a Schlenk flask equipped with a stir bar. The Schlenk flask was left under vacuum overnight. The Schlenk flask was purged with nitrogen and anhydrous pyridine was added (7.2 mL). The reaction was then cooled to 0° C. using an ice bath and stirred for 20 minutes before the dropwise addition of dimethylaluminum chloride (1.0 M in hexanes, 356 μL). The reaction was stirred at 0° C. for 30 minutes before removing the ice bath and letting the reaction warm to RT. The reaction was stirred for 48 hours. After 48 hours, the reaction was quenched with the addition of 30% w/v aq. tartaric acid (1.0 mL) at 0° C. and stirred for 1 min. The reaction warmed to RT and stirred for 5 minutes. The reaction mixture was diluted with ethyl acetate (20 mL), washed with water two times (20 mL), brine (20 mL), filtered through sodium sulfate and concentrated in vacuo. The residue was purified by chromatography on silica gel with a gradient of 0-100% EtOAc in hexanes to yield propan-2-yl (2S)-2-{[(S)-{[(4R,5R)-5-(2-amino-6-oxo-6,9-dihydro-1H-purin-9-yl)-4-hydroxy-4,5-dihydrofuran-2-yl]methoxy}(phenoxy)phosphoryl]amino}propanoate (205 mg, 40% yield). ¹H NMR (500 MHz, MeOD): 8.01 (s, 1H), 7.32 (t, J=7.0 Hz, 2H), 7.20 (d, J=8.0 Hz, 2H), 7.16 (t, J=7.5 Hz, 1H), 6.30 (s, 1H), 5.39 (s, 1H), 5.36 (s, 1H), 4.93 (m, 1H), 4.71 (m, 2H), 3.88 (s, 1H), 2.74 (m, 1H), 1.32 (d, J=7.0 Hz, 3H), 1.21 (m, 12H), 0.88 (s, 9H), 0.06 (s, 3H), 0.05 (s, 3H) δ ppm. ¹³C (126 MHz, MeOD): 181.9, 174.4 (d, J_{C-P}=6.3 Hz, 1C), 157.9 (d, J_{C-P}=9.5 Hz, 1C), 157.4, 152.1 (d, J_{C-P}=8.5 Hz, 1C), 150.3, 150.2, 138.9, 130.7, 126.2, 121.5 (d, J_{C-P}=5.9 Hz, 1C), 121.2, 104.2, 93.3, 81.4, 70.1, 61.8 (d, J_{C-P}=5.6 Hz, 1C), 51.7, 36.9, 26.2, 22.0, 21.9, 20.4 (d, J_{C-P}=8.6 Hz, 1C), 19.4, 19.3, 18.9, −4.4, −4.6 δ ppm. ³¹P NMR (202 NMR, CD₃OD): 3.74 δ ppm. HRMS (ESI) m z: calc'd for C₃₂H₄₆N₆O₉PSi [M-H]⁻: 717.8123; found 717.2743.

### g. Isopropyl ((S)-(((4R,5R)-5-(2-amino-6-oxo-1,6-dihydro-9H-purin-9-yl)-4-((tert-butyldimethylsilyl)oxy)-4,5-dihydrofuran-2-yl)methoxy)(phenoxy)phosphoryl)-L-alaninate

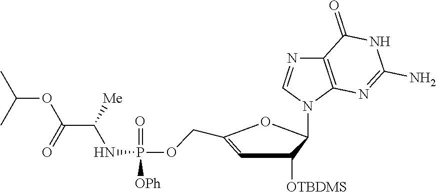

Propan-2-yl (2S)-2-{[(S)-{[(4R,5R)-5-(2-amino-6-oxo-6,9-dihydro-1H-purin-9-yl)-4-hydroxy-4,5-dihydrofuran-2-yl]methoxy}(phenoxy)-phosphoryl]amino}propanoate (275 mg, 383 mol) was dissolved in a solution of methylamine in THE (2.0 M solution, 3.83 mL). This was stirred overnight. The reaction was filtered with CH₃OH (15 mL). The filtrate was concentrated in vacuo. The residue was purified by chromatography on silica gel with a gradient of 0-20% CH₃OH in CH₂Cl₂ to yield isopropyl ((S)-(((4R,5R)-5-(2-amino-6-oxo-1,6-dihydro-9H-purin-9-yl)-4-((tert-butyldimethylsilyl)oxy)-4,5-dihydrofuran-2-yl)methoxy)(phenoxy)-phosphoryl)-L-alaninate (151 mg, 84% yield). ¹H NMR (500 MHz, MeOD): 7.76 (s, 1H), 7.31, (t, J=8.0 Hz, 2H), 7.21 (d, J=8.0 Hz, 2H), 7.16 (t, J=8.0 Hz, 1H), 6.23 (d, J=2.0 Hz, 1H), 5.38 (s, 1H), 5.34 (s, 1H), 4.94 (m, 1H), 4.71 (d, J=7.5 Hz, 2H), 3.90 (m, 1H), 1.33 (d, J=7.5 Hz, 3H), 1.20 (d, J=6.0 Hz, 6H), 0.88 (s, 9H), 0.06 (s, 6H) δ ppm. ¹³C (126 MHz, CD₃OD): 174.4 (d, J_{C-P}=6.5 Hz, 1C), 159.3, 157.7 (d, J_{C-P}=10.3 Hz, 1C), 155.6, 152.9, 152.1 (d, J_{C-P}=8.6 Hz, 1C), 136.8, 130.7, 126.2, 121.4 (d, J_{C-P}=5.9 Hz, 1C), 117.5, 104.2, 93.0, 81.3, 70.2, 61.9 (d, J_{C-P}=5.7 Hz, 1C), 51.7, 26.2, 22.0, 21.9, 20.4 (d, J_{C-P}=8.2 Hz, 1C), 18.9, −4.5, −4.6 δ ppm. ³¹P NMR (202 NMR, CD₃OD): 3.65 δ ppm. HRMS (ESI) m/z: calc'd for C₂₈H₄₀N₆O₈PSi [M-H]⁻: 647.2420; found 647.2331.

### h. Isopropyl ((S)-(((4R,5R)-5-(2-amino-6-oxo-1,6-dihydro-9H-purin-9-yl)-4-hydroxy-4,5-dihydrofuran-2-yl)methoxy)(phenoxy)phosphoryl)-L-alaninate

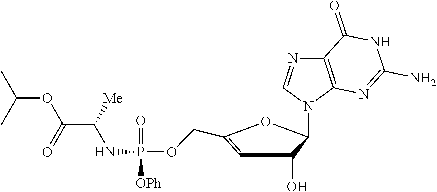

Isopropyl ((S)-(((4R,5R)-5-(2-amino-6-oxo-1,6-dihydro-9H-purin-9-yl)-4-((tert-butyldimethylsilyl)oxy)-4,5-dihydrofuran-2-yl)methoxy)(phenoxy)phosphoryl)-L-alaninate (161 mg, 248 mol) was dissolved in THF (1.83 mL). To this was added a 1:1 mixture of acetic acid:tetrabutylammonium fluoride (298 μL:298 μL). The reaction was stirred overnight. The reaction was concentrated in vacuo then dissolved in a 1:1 mixture of CH₃CN: H₂O (1.5 mL: 1.5 mL). The compound was purified via preparative HPLC (column: Agilent Technologies Zorbax SB-C18; 21.2×250 mm size, 7 m pore; method: solvent A, H₂O; solvent B, CH₃CN; gradient, 90:10 A:B from 0 to 2 min, followed by a linear gradient to 30:70 A:B from 2 to 22 min, followed by a linear gradient to 5:95 A:B from 22 to 28 min, and an isocratic 5:95 A:B from 28-35 min). Isopropyl ((S)-(((4R,5R)-5-(2-amino-6-oxo-1,6-dihydro-9H-purin-9-yl)-4-hydroxy-4,5-dihydrofuran-2-yl)methoxy)(phenoxy)phosphoryl)-L-alaninate was recovered (37 mg, 28% yield). ¹H NMR (500 MHz, DMSO-d₆): 10.71 (s, 1H), 7.67 (s, 1H), 7.34 (t, J=8.0 Hz, 2H), 7.17 (m, 3H), 6.57 (s, 2H), 6.06 (m, 2H), 5.83 (d, J=6.0 Hz, 1H), 5.39 (d, J=3.0 Hz, 1H), 5.12 (m, 1H), 4.83 (m, 1H), 4.60 (d, J=8.0 Hz, 2H), 3.77 (m, 1H), 1.21 (d, J=7.5 Hz, 3H), 1.13 (m, 6H) δ ppm. ¹³C (126 MHz, DMSO-d₆): 172.6 (d, J_{C-P}=6.3 Hz, 1C), 156.7, 155.2 (d, J_{C-P}=10.7 Hz, 1C), 154.0, 151.0, 150.6 (d, J_{C-P}=7.7 Hz, 1C), 134.3, 129.6, 124.6, 120.1 (d, J_{C-P}=5.4 Hz, 1C), 116.2, 103.4, 90.6, 77.4, 68.0, 60.0 (d, J_{C-P}=5.4 Hz, 1C), 49.8, 21.4, 21.4, 19.6 (d, J_{C-P}=8.0 Hz, 1C) δ ppm. ³¹P NMR (202 NMR, DMSO-d₆): 3.54 δ ppm. HRMS (ESI) m/z: calcd for C₂₂H₂₇N₆O₆P [M-H]⁻: 533.1555; found 533.1545.

### Example 5. Synthesis of Isopropan-2-yl (2S)-2-{[(S)-{[5-(6-amino-9H-purin-9-yl)-4-hydroxy-4,5-dihydrofuran-2-yl]methoxy}-(phenoxy)phosphoryl]amino}propanoate

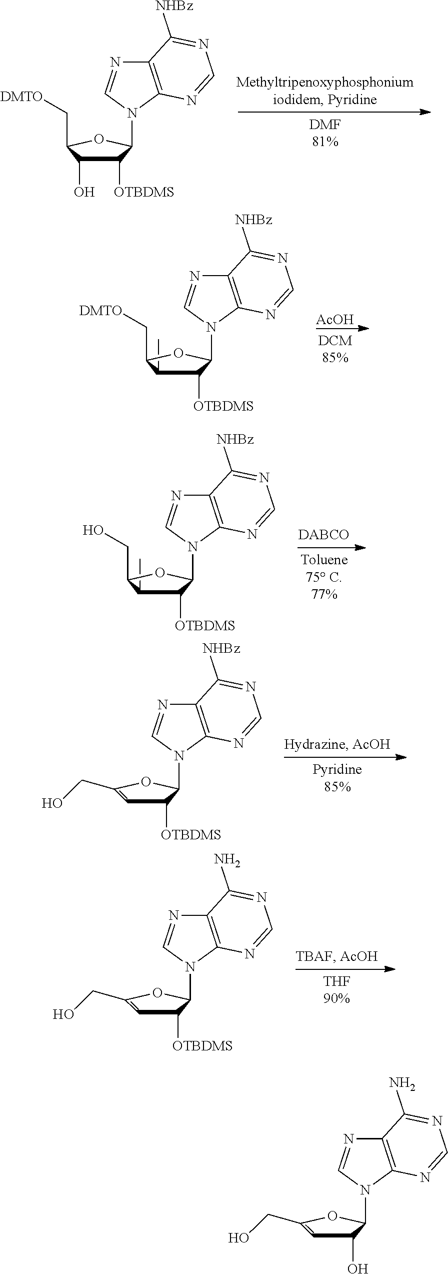

### a. 5′-DMT-3′-Iodo-2′-TBDMS-N-Bz-adenosine

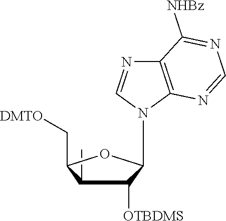

N-[9-(5-{[bis(4-methoxyphenyl)(phenyl)methoxy]methyl}-3-[(tert-butyldimethy-lsilyl)oxy]-4-hydroxyoxolan-2-yl)-9H-purin-6-yl]benzamide (788 mg, 1.00 mmol) was placed in a Schlenk flask and placed on the high-vacuum for one hour before the addition of methyltriphenoxyphosphonium iodide (724 mg, 1.60 mmol). The flask was then purged and filled with nitrogen 3 times before the addition of solvent. To the purged flask was added DMF (6.6 mL) and then pyridine (160 μL, 1.32 mmol). The reaction was stirred under nitrogen overnight (18 hours). After 18 hours, the reaction was quenched by the addition of CH₃OH (0.5 mL) and TEA (0.5 mL) and stirred for 15 minutes. The reaction mixture was diluted with ethyl acetate (20 mL), washed with water two times (20 mL), brine (20 mL), filtered through sodium sulfate and concentrated in vacuo. The residue was purified by chromatography on silica gel with a gradient of 0-30% EtOAc in hexanes to yield 5′-DMT-3′-Iodo-2′-TBDMS-N-Bz-adenosine (728 mg, 81% yield). ¹H NMR (500 MHz, DMSO-d₆): 11.23 (s, 1H), 8.69 (s, 1H), 8.48 (s, 1H), 8.05 (d, J=8.0 Hz, 2H), 7.65 (t, J=7.5 Hz, 1H), 7.55 (t, J=8.0 Hz, 2H), 7.43 (d, J=8.0 Hz, 2H), 7.29 (m, 6H), 7.24 (d, J=7.0 Hz, 1H), 6.86 (m, 4H), 6.03 (d, J=5.0 Hz, 1H), 5.36 (t, J=5.0 Hz, 1H), 4.54 (t, J=6.5 Hz, 1H), 4.30 (m, 1H), 3.73 (s, 6H), 3.54 (m, 1H), 0.79 (s, 9H), 0.10 (s, 3H), −0.22 (s, 3H) δ ppm. ¹³C (126 MHz, DMSO-d₆): 168.1, 160.3, 153.4, 153.2, 151.2, 146.1, 143.8, 136.8, 136.8, 135.0 133.9, 131.6, 131.5, 129.8, 129.6, 129.4, 128.8, 128.1, 125.2, 114.1, 92.0, 88.2, 84.0, 81.6, 70.1, 55.7, 28.2, 26.1, 18.7, −4.1, −4.7 δ ppm. HRMS (ESI) m z: calc'd for C₄₄H₄₇IN₅O₆Si [M-H]⁻: 896.2346; found 896.2223.

### b. 3′-Iodo-2′-TBDMS-N-Bz-adenosine

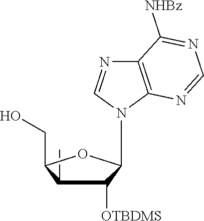

5′-DMT-3′-Iodo-2′-TBDMS-N-Bz-adenosine (2.81 g, 3.13 mmol) was dissolved in CH₂Cl₂ (1.54 mL) and to this solution was added a 4:1 mixture of acetic acid:water (3.13 mL: 772 μL). The reaction mixture was stirred overnight. The reaction mixture was diluted with ethyl acetate (30 mL), washed with water two times (30 mL), brine (30 mL), filtered through sodium sulfate and concentrated in vacuo. The residue was purified by chromatography on silica gel with a gradient of 0-30% EtOAc in hexanes to yield 3′-Iodo-2′-TBDMS-N-Bz-adenosine (728 mg, 85% yield). ¹H NMR (500 MHz, CDCl₃): 8.84 (s, 1H), 8.13 (s, 1H), 8.04 (m, 2H), 7.63 (t, J=7.5 Hz, 1H), 7.54 (t, J=8.0 Hz, 2H), 5.68 (d, J=6.0 Hz, 1H), 5.25 (m, 1H), 4.47 (t, J=8.0 Hz, 1H), 4.39 (m, 1H), 4.13 (d, J=2.0 Hz, 2H), 0.81 (s, 9H), 0.08 (s, 3H), −0.50 (s, 3H) δ ppm. ¹³C (126 MHz, CDCl₃): 164.5, 152.7, 150.6, 150.5, 143.0, 133.6, 133.2, 129.1, 128.0, 124.2, 91.1, 81.0, 79.1, 66.1, 25.7, 24.1, 17.9, −3.6, −5.2 δ ppm. HRMS (ESI) m/z: calc'd for C₂₃H₂₉IN₅O₄Si [M-H]⁻: 594.1039; found 594.0960.

### c. 3′-Deoxy-3′,4′-didehydro-2′-TBDMS-N-Bz-adenosine

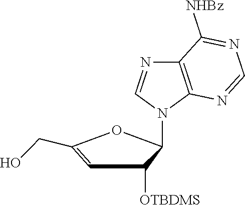

3′-Iodo-2′-TBDMS-N-Bz-adenosine (1.59 g, 2.67 mmol) was suspended in toluene (15.0 mL) and to the solution was added DABCO (1.05 mg, 9.34 mmol). The reaction mixture was then heated to 75° C. and stirred overnight (18 hours). The reaction mixture was cooled and filtered to remove the salts formed. The collected filtrate was concentrated in vacuo. The residue was purified by chromatography on silica gel with a gradient of 0-100% EtOAc in hexanes to yield 3′-Deoxy-3′,4′-didehydro-2′-TBDMS-N-Bz-adenosine (960 mg, 77% yield). ¹H NMR (500 MHz, MeOD): 8.75 (s, 1H), 8.45 (s, 1H), 8.09 (d, J=7.5 Hz, 2H), 7.66 (t, J=7.5 Hz, 1H), 7.57 (t, J=8.0 Hz, 2H), 6.54 (d, J=2.0 Hz, 1H), 5.42 (s, 1H), 5.38 (m, 1H), 4.22 (s, 2H), 0.92 (s, 9H), 0.11 (s, 3H), 0.10 (s, 3H) δ ppm. HRMS (ESI) m/z: calcd for C₂₂H₂₉N₅O₈P [M-H]518.1446; found 518.1436. ¹³C (126 MHz, CDCl₃): 168.2, 163.2, 153.6, 153.1, 151.3, 143.0, 134.9, 133.9, 129.8, 129.5, 125.0, 101.4, 93.6, 81.5, 57.9, 26.2, 18.9, −4.5 δ ppm. HRMS (ESI) m z: calc'd for C₂₃H₂₈N₅O₄Si [M-H]⁻: 466.1916; found 466.1854.

### d. 3′-Deoxy-3′,4′-didehydro-2′-TBDMS-adenosine

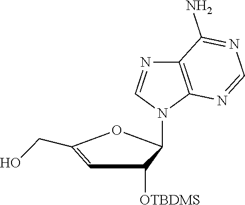

3′-Deoxy-3′,4′-didehydro-2′-TBDMS-N-Bz-adenosine (750 mg, 1.60 mmol) was dissolved in pyridine (4.90 mL) and acetic acid (1.22 mL). To this was added hydrazine hydrate (236 μL, 4.81 mmol). The reaction was stirred overnight. The reaction was concentrated in vacuo. The residue was purified by chromatography on silica gel with a gradient of 0-10% CH₃OH in CH₂Cl₂ to yield 3′-Deoxy-3′,4′-didehydro-2′-TBDMS-adenosine (493 mg, 85% yield). ¹H NMR (500 MHz, MeOD): 8.22 (s, 1H), 8.16 (s, 1H), 6.40 (d, J=2.0 Hz, 1H), 5.39 (s, 1H), 5.33 (m, 1H), 4.19 (m, 2H), 0.89 (s, 9H), 0.07 (s, 3H), 0.07 (s, 3H) δ ppm. ¹³C (126 MHz, CDCl₃): 163.1, 157.4, 154.3, 150.4, 139.9, 120.0, 101.2, 93.3, 81.6, 58.0, 26.2, 18.9, −4.5, −4.6 δ ppm. HRMS (ESI) m/z: calc'd for C₁₆H₂₄N₅O₃Si [M-H]⁻: 362.1654; found 362.1605.

### e. 3′-Deoxy-3′,4′-didehydro-adenosine

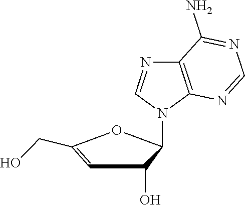

3′-Deoxy-3′,4′-didehydro-2′-TBDMS-adenosine (493 mg, 1.36 mmol) was dissolved in THF (9.8 mL). To this was added a 1:1 mixture of acetic acid: tetrabutylammonium fluoride (1.62 mL:1.62 mL). The reaction was stirred overnight. The product precipitated from the solution over the course of the reaction. The solid residue was filtered with EtOAc (20 mL) and dried in vacuo. This yielded 3′-Deoxy-3′,4′-didehydro-adenosine (305 mg, 90% yield). ¹H NMR (500 MHz, DMSO-d₆): 8.17 (s, 1H), 8.14 (s, 1H), 7.33 (s, 2H), 6.26 (d, J=2.0 Hz, 1H), 5.73 (d, J=6.0 Hz, 1H), 5.23 (m, 3H), 4.01 (d, J=5.5 Hz, 2H) δ ppm. ¹³C (126 MHz, DMSO-d₆): 116.0, 156.1, 153.0, 149.1, 138.4, 118.6, 99.9, 91.3, 77.3, 56.2 δ ppm. HRMS (ESI) m/z: calc'd for C₁₀H₁₀N₅O₃ [M-H]⁻: 248.0789; found 248.0754.

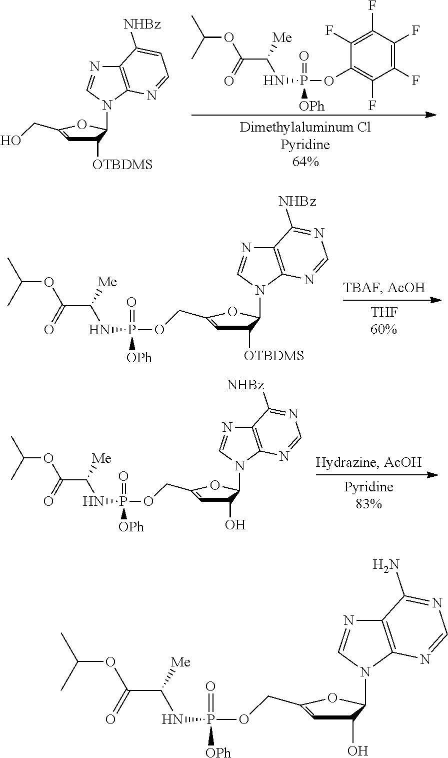

### f. Propan-2-yl (2S)-2-{[(S)-{[5-(6-benzamido-9H-purin-9-yl)-4-[(tert-butyldimethylsilyl)oxy]-4,5-dihydrofuran-2-yl]methoxy}(phenoxy)phosphoryl]-amino}propanoate

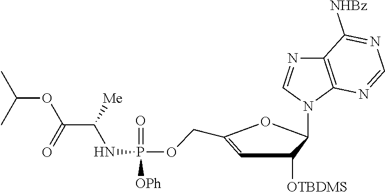

3′-Deoxy-3′,4′-didehydro-2′-TBDMS-N-Bz-adenosine (329 mg, 704 mol) and propan-2-yl (2S)-2-{[(2,3,4,5,6 pentafluoro-phenoxy)(phenoxy)phosphoryl]amino}-propanoate (478 mg, 1.06 mmol) were placed in a Schlenk flask equipped with a stir bar. The Schlenk flask was left under vacuum overnight. The Schlenk flask was purged with nitrogen and anhydrous pyridine was added (6.97 mL). The reaction was then cooled to 0° C. using an ice bath and stirred for 20 minutes before the dropwise addition of dimethylaluminum chloride (1.0 M in hexanes, 352 μL). The reaction was stirred at 0° C. for 30 minutes before removing the ice bath and letting the reaction warm to RT. The reaction was stirred for 48 hours. After 48 hours, the reaction was quenched with the addition of 30% w/v aq. tartaric acid (1.0 mL) at 0° C. and stirred for 1 min. The reaction warmed to RT and stirred for 5 minutes. The reaction mixture was diluted with ethyl acetate (20 mL), washed with water two times (20 mL), brine (20 mL), filtered through sodium sulfate and concentrated in vacuo. The residue was purified by chromatography on silica gel with a gradient of 0-100% EtOAc in hexanes to yield propan-2-yl (2S)-2-{[(S)-{[5-(6-benzamido-9H-purin-9-yl)-4-[(tert-butyldimethylsilyl)oxy]-4,5-dihydrofuran-2-yl]methoxy}(phenoxy)phosphoryl]-amino}propanoate (334 mg, 64% yield). ¹H NMR (500 MHz, CDCl₃): 8.82 (s, 1H), 8.12 (s, 1H), 8.04 (d, J=7.5 Hz, 2H), 7.62 (t, J=7.5 Hz, 1H), 7.53 (t, J=8.0 Hz, 2H), 7.28 (t, J=8.0 Hz, 2H), 7.20 (d, J=8.5 Hz, 2H), 7.13 (t, J=7.0 Hz, 1H), 6.47 (d, J=2.5 Hz, 1H), 5.33 (s, 1H), 4.98 (m, 1H), 4.72 (d, J=8.5 Hz, 2H), 3.98 (m, 1H), 3.73 (t, J=10.5 Hz, 1H), 1.37 (d, J=7.5 Hz, 3H), 1.21 (d, J=6.5 Hz, 6H), 0.88 (s, 9H), 0.06 (s, 3H), 0.04 (s, 3H) δ ppm. ¹³C (126 MHz, CD₃OD): 174.4 (d, J_{C-P}=6.5 Hz, 1C), 168.2, 157.8 (d, J_{C-P}=9.8 Hz, 1C), 153.7, 153.1, 152.1 (d, J_{C-P}=8.5 Hz, 1C), 151.4, 143.3, 134.9, 133.9, 130.7, 129.8, 129.5, 126.2, 125.0, 121.4 (d, J_{C-P}=5.7 Hz, 1C), 104.7, 93.8, 81.2, 70.1, 61.8 (d, J_{C-P}=5.9 Hz, 1C), 51.7, 26.2, 22.0, 21.9, 20.4 (d, J_{C-P}=8.2 Hz, 1C), 18.9, −4.4, −4.6 δ ppm. ³¹P NMR (202 NMR, CD₃OD): 3.54 δ ppm. HRMS (ESI) m/z: calc'd for C₃₅H₄₄N₆O₈Psi [M-H]⁻: 735.2733; found 735.2634.

### g. Propan-2-yl (2S)-2-{[(S)-{[5-(6-benzamido-9H-purin-9-yl)-4-hydroxy-4,5-dihydrofuran-2-yl]methoxy}(phenoxy)phosphoryl]-amino}propanoate

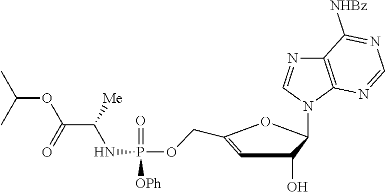

Propan-2-yl (2S)-2-{[(S)-{[5-(6-benzamido-9H-purin-9-yl)-4-[(tert-butyldimethyl-silyl)oxy]-4,5-dihydrofuran-2-yl]methoxy}(phenoxy)phosphoryl]-amino}propanoate (326 mg, 442 mol) was dissolved in THE (3.24 mL). To this was added a 1:1 mixture of acetic acid: tetrabutylammonium fluoride (533 μL:533 μL). The reaction was stirred overnight. The reaction was concentrated in vacuo then dissolved in a 1:1 mixture of CH₃CN:H₂O (1.5 mL: 1.5 mL). The reaction was concentrated in vacuo. The residue was purified by chromatography on silica gel with a gradient of 0-15% CH₃OH in CH₂Cl₂ to yield propan-2-yl (2S)-2-{[(S)-{[5-(6-benzamido-9H-purin-9-yl)-4-hydroxy-4,5-dihydrofuran-2-yl]methoxy}(phenoxy)phosphoryl]-amino}propanoate (165 mg, 60% yield). ¹H NMR (500 MHz, MeOD): 8.74 (s, 1H), 8.43 (s, 1H), 8.09 (d, J=7.5 Hz, 2H), 7.67 (t, J=7.0 Hz, 1H), 7.57 (t, J=8.0 Hz, 2H), 7.28 (t, J=8.5 Hz, 2H), 7.18 (d, J=9.0 Hz, 2H), 7.13 (t, J=7.5 Hz, 1H), 6.56 (d, J=2.5 Hz, 1H), 5.55 (d, J=2.0 Hz, 1H), 5.33 (s, 1H), 4.92 (m, 1H), 4.76 (m, 2H), 3.89 (m, 1H), 1.33 (d, J=7.0 Hz, 3H), 1.19 (m, 6H) δ ppm. ¹³C (126 MHz, CD₃OD): 174.4 (d, J_{C-P}=6.5 Hz, 1C), 168.1, 157.8 (d, J_{C-P}=9.7 Hz, 1C), 153.6, 153.1, 152.1 (d, J_{C-P}=8.5 Hz, 1C), 151.3, 143.3, 135.0, 133.9, 130.7, 129.8, 129.5, 126.1, 124.9, 121.3 (d, J_{C-P}=5.7 Hz, 1C), 104.5, 93.6, 79.6, 70.1, 61.8 (d, J_{C-P}=5.7 Hz, 1C), 51.7, 22.0, 21.9, 20.4 (d, J_{C-P}=7.9 Hz, 1C) δ ppm. ³¹P NMR (202 NMR, CD₃OD): 3.65 δ ppm. HRMS (ESI) m/z: calc'd for C₂₉H₃₀N₆O₈P [M-H]⁻: 621.1868; found 621.1784.

### h. Isopropan-2-yl (2S)-2-{[(S)-{[5-(6-amino-9H-purin-9-yl)-4-hydroxy-4,5-dihydrofuran-2-yl]methoxy}-(phenoxy)phosphoryl]amino}propanoate

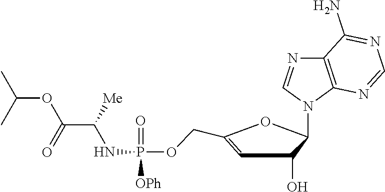

Propan-2-yl (2S)-2-{[(S)-{[5-(6-benzamido-9H-purin-9-yl)-4-hydroxy-4,5-dihydrofuran-2-yl]methoxy}(phenoxy)phosphoryl]-amino}propanoate (152 mg, 244 mol) was dissolved in pyridine (740 μL) and acetic acid (185 μL). This was stirred overnight. The reaction was concentrated in vacuo. The residue was purified by chromatography on silica gel with a gradient of 0-15% CH₃OH in CH₂Cl₂ to yield isopropyl propan-2-yl (2S)-2-{[(S)-{[5-(6-amino-9H-purin-9-yl)-4-hydroxy-4,5-dihydrofuran-2-yl]methoxy}(phenoxy)phosphoryl]amino}propanoate (105 mg, 83% yield). ¹H NMR (500 MHz, DMSO-d₆): 8.18 (s, 1H), 8.17 (s, 1H), 7.35 (s, 2H), 7.30 (t, J=7.5 Hz, 2H), 7.15 (m, 3H), 6.30 (d, J=2.5 Hz, 1H), 6.04 (m, 1H), 5.87 (d, J=5.5 Hz, 1H), 5.44 (d, J=2.5 Hz, 1H), 5.30 (m, 1H), 4.81 (m, 1H), 4.61 (d, J=7.5 Hz, 2H), 3.77 (m, 1H), 1.21 (d, J=7.0 Hz, 3H), 1.11 (t, J=6.5 Hz, 6H) δ ppm. ¹³C (126 MHz, DMSO-d₆): 172.5 (d, J_{C-P}=6.2 Hz, 1C), 156.1, 155.0 (d, J_{C-P}=10.9 Hz, 1C), 153.0, 150.6 (d, J_{C-P}=7.9 Hz, 1C), 149.1, 138.7, 129.6, 124.6, 120.1 (d, J_{C-P}=5.9 Hz, 1C), 118.7, 103.7, 91.5, 77.2, 68.0, 60.0 (d, J_{C-P}=5.4 Hz, 1C), 49.8, 21.4, 21.3, 19.6 (d, J_{C-P}=8.0 Hz, 1C) δ ppm. ³¹P NMR (202 NMR, DMSO-d₆): 3.53 δ ppm. HRMS (ESI) m/z: calcd for C₂₂H₂₇N₆O₇P [M-H]⁻: 517.1606; found 517.1596.

### Example 5. Biological Testing

HUH7 and Vero cell lines were maintained in a humidified 5% CO₂ environment at 37° C. HUH7 cells were cultured in DMEM (4500 mg/L “high glucose”+L-glutamine+25 mM HEPES-sodium pyruvate) supplemented with fetal bovine serum (10% FBS), penicillin (100 IU/mL), streptomycin (100 g/mL), and 1% nonessential amino acids (NEAA). Vero cells were cultured in EMEM media supplemented with FBS (10% FBS), penicillin (100 IU/mL), and streptomycin (100 μg/mL). Cytotoxicity assays were done similarly to a previous report (J. Med. Chem. 2021, 64, 15429-15439). HUH7 or Vero cells were seeded at 2,000 cells/well in their appropriate cell culture media (50 μL) in 96-well clear plates and incubated for 24 hours. A DMSO solution of each individual compound was serially diluted (solution concentrations of 0.1, 0.5 and 1.0 mM were made) in the appropriate cell line media, and each solution (50 μL) was dosed to the cells (well volume total of 100 μL; final DMSO concentration of 0.5%). DMSO-only and no-cell control wells were dosed with a DMSO-media solution (50 μL of 1% DMSO in media) rather than compound. The cells were then incubated for 2 or 5 days. Two hours before the designated time point, Alamar blue (10 μL) was added to each well. At the time point, fluorescence data were measured using a BioTek Synergy H1 microplate reader. Cell viability was calculated by subtracting background fluorescence (no-cell control) from the measured fluorescence of each well. Wells with compounds were normalized to the DMSO-only control (100% viability) and no-cell controls. Each individual experiment was performed in groups of three technical replicates. For the reported Vero studies, cytotoxicity experiments were repeated in biological triplicate. The final cell viability percentage was calculated by averaging % viability from each of the biological replicates, and the uncertainty was calculated by propagating the standard deviations from each biological replicate (taking the square root of the sum of the squares of individual standard deviations).

### Results

The following compounds are discussed below.

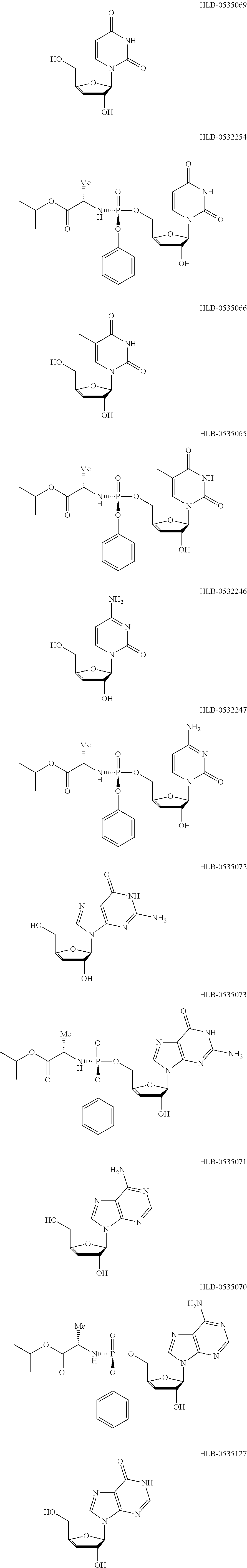

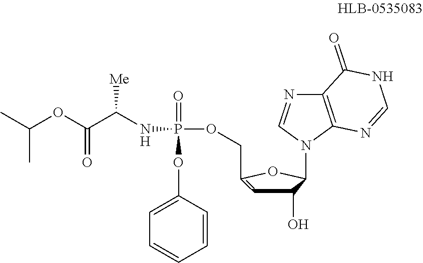

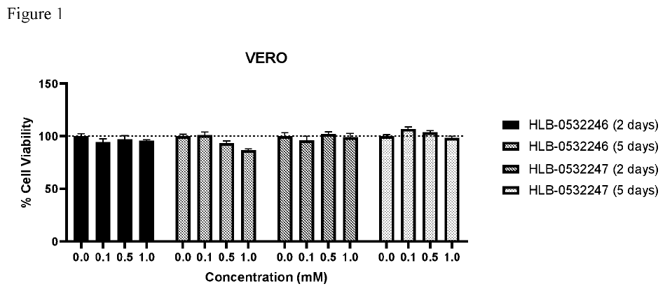

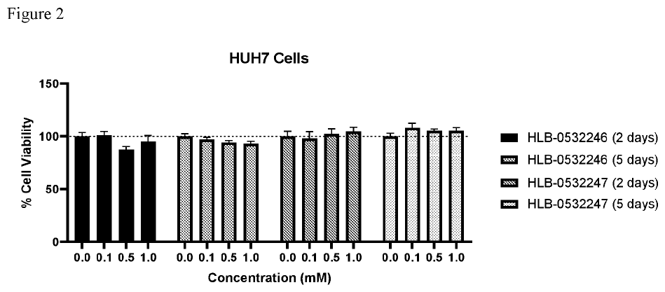

HLB-0532246 showed a 10% decrease in cell viability when dosed at high concentrations after 5 days in Vero and HUH7 cells. HLB-0532247 did not show any cytotoxicity after 2 or 5 days in both Vero (See FIG. 1) and HUH7 cells (See FIG. 2).

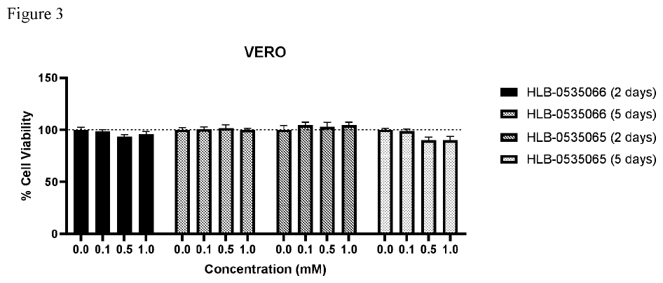

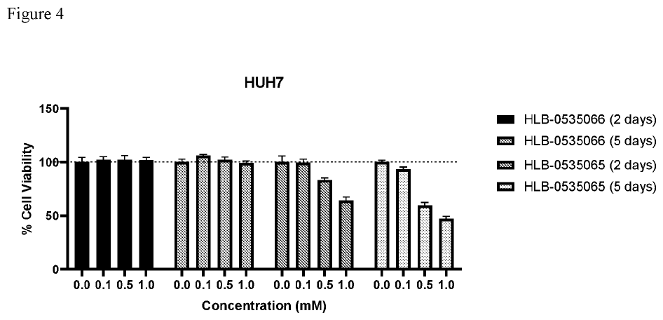

Compounds HLB-0535065 and HLB-0535066 did not show any cytotoxicity in Vero cells after 2 or 5 days (FIG. 3), however in HUH7 cells the cell viability decreased to 50% by day 5 in cells dosed with HLB-0535065 (FIG. 4). HUH7 cells dosed with HLB-0535066 remained unchanged (FIG. 4).

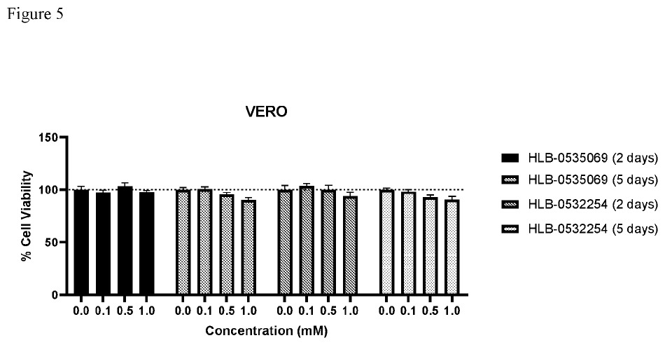

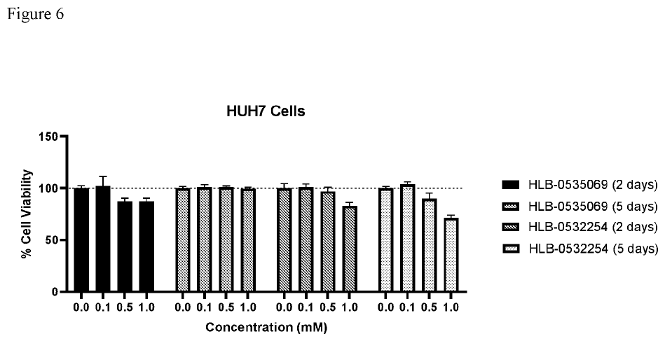

In both Vero and HUH7 cells compound HLB-0535069 did not show a significant decrease in cell viability over 5 days (FIGS. 5 and 6), whereas with compound HLB-0532254 a 10% decrease in cell viability was determined for Vero cells (FIG. 5) and a 25% decrease in cell viability was determined for HUH7 cells (FIG. 6).

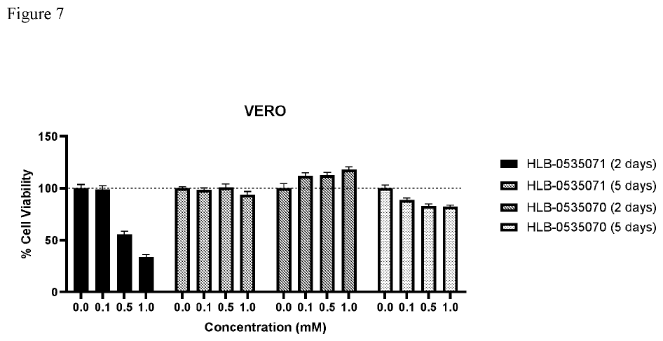

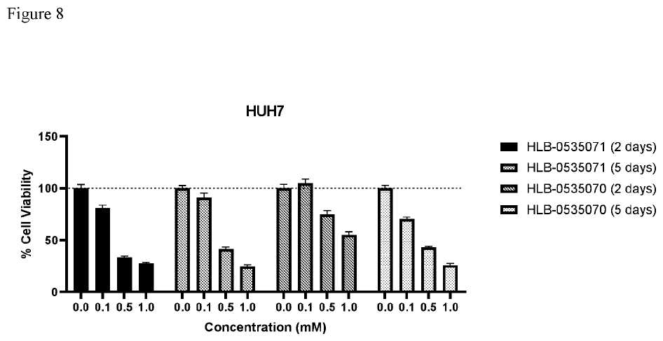

When HLB-0535070 was dosed in Vero cells, it showed a 30% decrease in cell viability after 5 days, whereas with HLB-0535071 a significant decrease in cell viability (75%) after 5 days was observed (FIG. 7). Interestingly, in HUH7 cells, both HLB-0535070 and HLB-0535071 led to significant cell death over 2 and 5 days (FIG. 8).

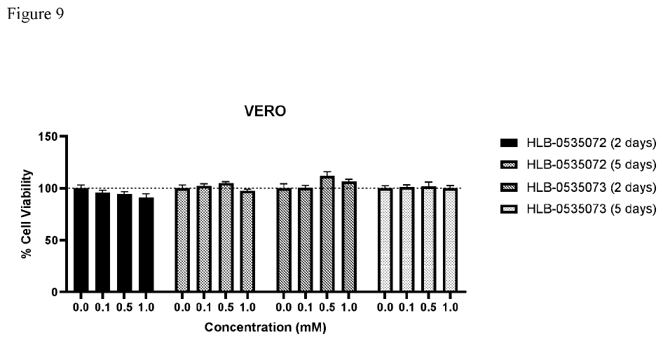

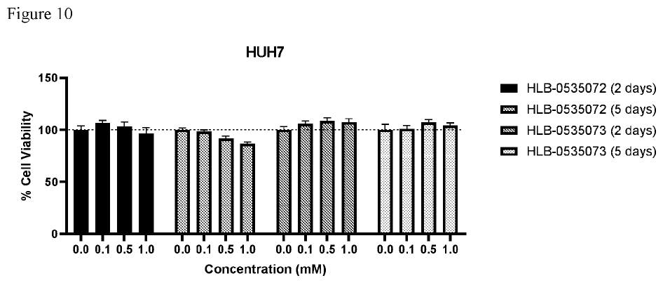

No significant change in cell viability was observed in Vero cells dosed with HLB-0535073 or HLB-0535072 (FIG. 9). No significant change in cell viability was observed in HUH7 cells dosed with HLB-0535073, whereas a slight decrease in cell viability was observed with HLB-0535072 but only after 5 days (FIG. 10).

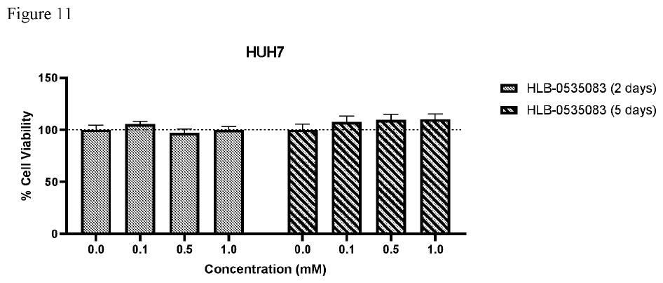

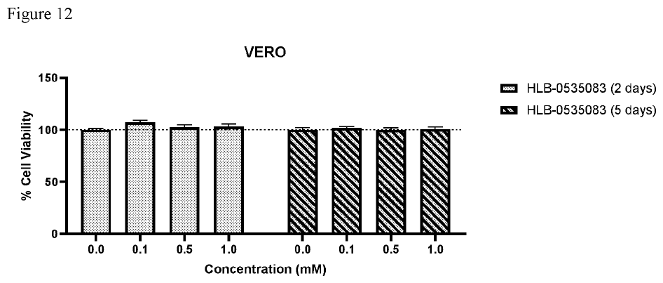

No change in cell viability was observed in HUH7 cells dosed with HLB-0535083 after 5 days (FIG. 11) or in Vero cells dosed with HLB-0535083 after 5 days (FIG. 12).

The compounds HLB-0532246 and HLB-0532247 have been previously tested for antiviral activity. HLB-0532246 demonstrated antiviral activity against ZIKV in Vero cells (Gizzi, A. S, et al., Nature 2018, 558, 610). However, this compound was inactive when tested against WNV. HLB-0532247 showed potent antiviral activity against ZIKV in both Vero and HUH7 cells and WNV in HUH7 cells (Passow, K. T., et al., J Med. Chem. 2021, 64, 15429).

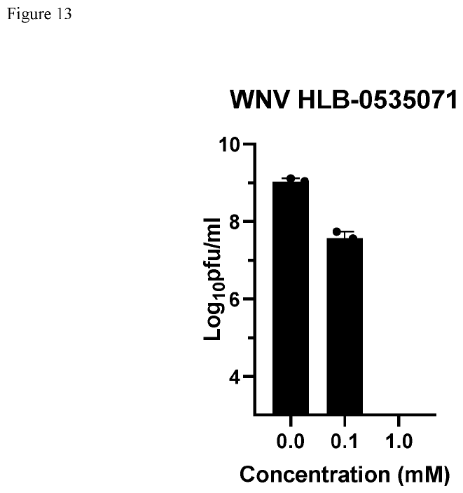

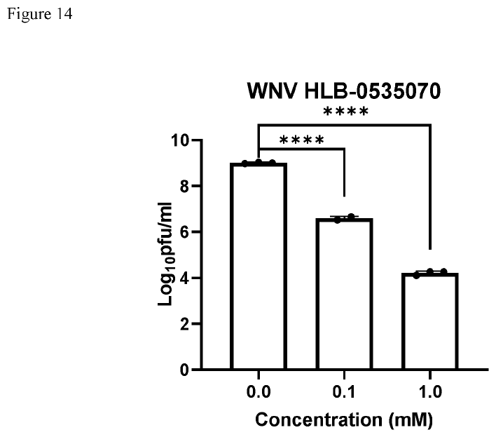

The antiviral plaque assay with West Nile Virus showed no activity from the free nucleosides (HLB-0535066, HLB-0535069, HLB-0535127, or HLB-0535072) or prodrugs (HLB-0535065, HLB-0532254, HLB-0535083, or HLB-0535073). However, potent antiviral activity was observed from both HLB-0535071 (FIG. 13) and HLB-0535070 (FIG. 14).

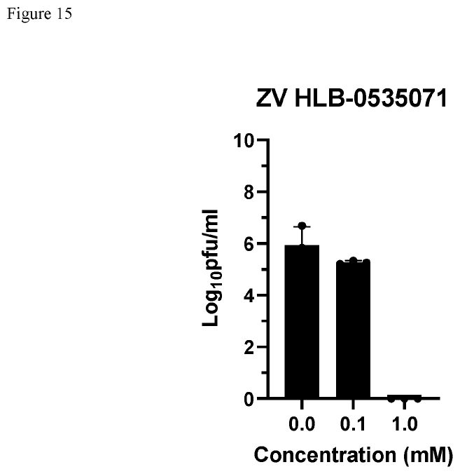

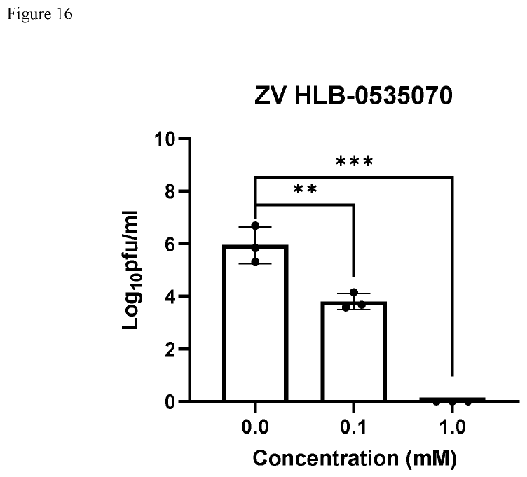

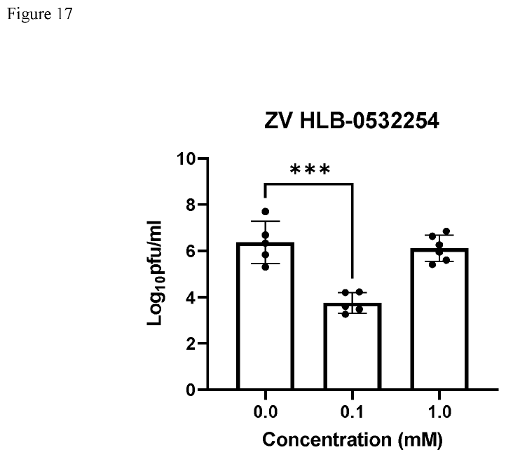

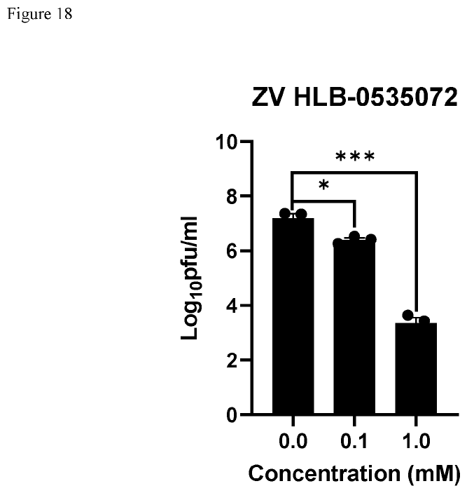

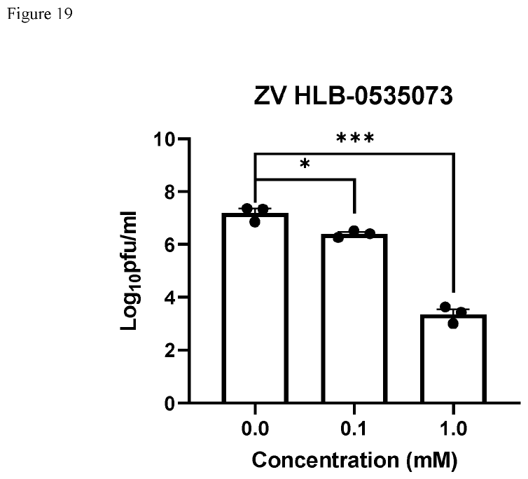

The antiviral testing against ZIKV showed no activity from the free nucleosides or prodrugs HLB-0535066, HLB-0535065, HLB-0535127 and HLB-0535083. Similar to what was previously observed in WNV, HLB-0535071 (FIG. 15) and HLB-0535070 (FIG. 16) were observed to have potent antiviral activity. Although not active against WNV, HLB-0532254 showed potent activity against ZIKV when dosed at 0.1 mM (FIG. 17); however, the prodrug did not display antiviral activity at 1.0 mM. The free nucleoside HLB-0535069 did not display antiviral activity. Antiviral activity was observed from both the free nucleoside HLB-0535072 (FIG. 18) and prodrug HLB-0535073 (FIG. 19) against ZIKV.

### Example 6

The following illustrate representative pharmaceutical dosage forms, containing a compound of formula I (‘Compound X’), for therapeutic or prophylactic use in humans.

| (i) Tablet 1 | mg/tablet |
|---|---|
| Compound X= | 100.0 |
| Lactose | 77.5 |
| Povidone | 15.0 |
| Croscarmellose sodium | 12.0 |
| Microcrystalline cellulose | 92.5 |
| Magnesium stearate | 3.0 |
| | 300.0 |

| (ii) Tablet 2 | mg/tablet |
|---|---|
| Compound X= | 20.0 |
| Microcrystalline cellulose | 410.0 |
| Starch | 50.0 |
| Sodium starch glycolate | 15.0 |
| Magnesium stearate | 5.0 |
| | 500.0 |

| (iii) Capsule | mg/capsule |
|---|---|
| Compound X= | 10.0 |
| Colloidal silicon dioxide | 1.5 |
| Lactose | 465.5 |
| Pregelatinized starch | 120.0 |
| Magnesium stearate | 3.0 |
| | 600.0 |

| (iv) Injection 1 (1 mg/ml) | mg/ml |
|---|---|
| Compound X = (free acid form) | 1.0 |
| Dibasic sodium phosphate | 12.0 |
| Monobasic sodium phosphate | 0.7 |
| Sodium chloride | 4.5 |
| 1.0N Sodium hydroxide solution | q.s. |
| (pH adjustment to 7.0-7.5) | |
| Water for injection | q.s. ad 1 mL |

| (v) Injection 2 (10 mg/ml) | mg/ml |
|---|---|
| Compound X = (free acid form) | 10.0 |
| Monobasic sodium phosphate | 0.3 |
| Dibasic sodium phosphate | 1.1 |
| Polyethylene glycol 400 | 200.0 |
| 1.0N Sodium hydroxide solution | q.s. |
| (pH adjustment to 7.0-7.5) | |
| Water for injection | q.s. ad 1 mL |

| (vi) Aerosol | mg/can |
|---|---|
| Compound X= | 20.0 |
| Oleic acid | 10.0 |
| Trichloromonofluoromethane | 5,000.0 |
| Dichlorodifluoromethane | 10,000.0 |
| Dichlorotetrafluoroethane | 5,000.0 |

The above formulations may be obtained by conventional procedures well known in the pharmaceutical art.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

1. A compound of formula I 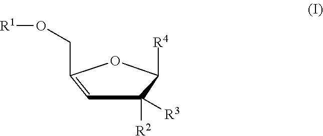
wherein:
R¹ is H, —P(═X)(OH)—O—P(═O)(OH)—P(═O)(OH)₂, or —P(═X)(R^{a})(R^{b});
R² is H, F, Cl, Br, OH, (C₁-C₃)alkyl or (C₁-C₃)alkoxy, wherein any (C₁-C₃)alkyl and (C₁-C₃)alkoxy is optionally substituted with one or more halo;
R³ is H, F, Cl, Br, OH, (C₁-C₃)alkyl or (C₁-C₃)alkoxy, wherein any (C₁-C₃)alkyl and (C₁-C₃)alkoxy is optionally substituted with one or more halo;
R⁴ is selected from the group consisting of: 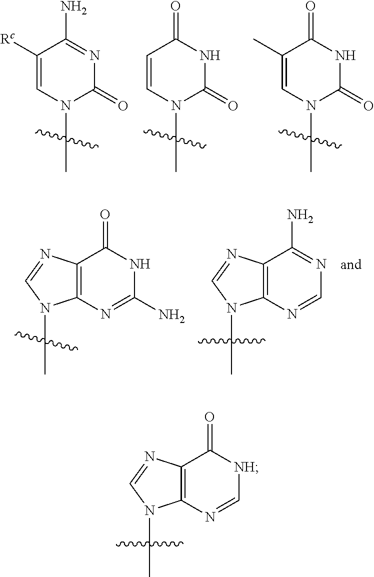
R^{a} is an N-linked amino acid or a (C₁-C₆)alkyl ester of an N-linked amino acid, wherein the (C₁-C₆)alkyl is optionally substituted with phenyl;
R^{b} is (C₁-C₆)alkoxy, aryloxy, or 5-10 membered heteroaryloxy, wherein any (C₁-C₆)alkoxy, aryloxy, and 5-10 membered heteroaryloxy is optionally substituted with one or more groups independently selected from the group consisting of halo, hydroxy, (C₁-C₃)alkoxy and (C₁-C₃)haloalkoxy;
R^{c} is (C₁-C₃)alkyl, hydroxymethyl, formyl or carboxy; and
X is S or O;
or a salt thereof.

2. The compound or salt of claim 1, wherein R¹ is H.

3. The compound or salt of claim 1, wherein R¹ is —P(═X)(OH)—O—P(═O)(OH)—P(═O)(OH)₂.
or —P(═X)(R^{a})(R^{b}).

4. The compound or salt of claim 1, wherein R² is F, Cl, Br, OH, (C₁-C₃)alkyl or (C₁-C₃)alkoxy, wherein any (C₁-C₃)alkyl and (C₁-C₃)alkoxy is optionally substituted with one or more (e.g., 1, 2, 3, or 4) halo.

5. The compound or salt of claim 1, wherein R² is H.

6. The compound or salt of claim 1, wherein R² is F, OH, or methoxy.

7. The compound or salt of claim 1, wherein R² is OH.

8. The compound or salt of claim 1, wherein R³ is F, Cl, Br, OH, (C₁-C₃)alkyl or (C₁-C₃)alkoxy, wherein any (C₁-C₃)alkyl and (C₁-C₃)alkoxy is optionally substituted with one or more halo.

9. The compound or salt of claim 1, wherein R³ is H.

10. The compound or salt of claim 1, wherein R³ is methyl or F.

11. The compound or salt of claim 1, wherein R⁴ is selected from the group consisting of:

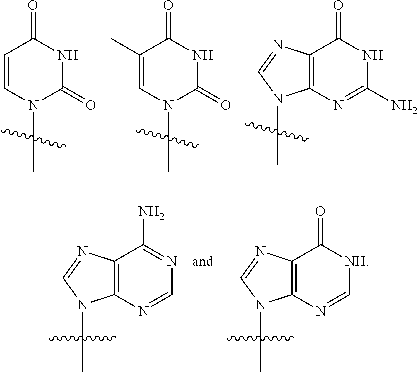

12-21. (canceled)

22. The compound or salt of claim 1, wherein R^{a} is R^{d}—O—C(═O)—CH(R^{e})—NH—; wherein R^{d} is (C₁-C₆)alkyl that is optionally substituted with phenyl; and R^{e} is the side-chain of a natural amino acid.

23-25. (canceled)

26. The compound or salt of claim 1, wherein R^{b} is phenoxy that is optionally substituted with one or more groups independently selected from the group consisting of halo, hydroxy, (C₁-C₃)alkoxy and (C₁-C₃)haloalkoxy.

27. (canceled)

28. The compound or salt of claim 1, wherein R^{c} is methyl, hydroxymethyl, formyl or carboxy.

29-34. (canceled)

35. The compound or salt of claim 1, wherein the compound of formula (I) is a compound of formula (Ic):

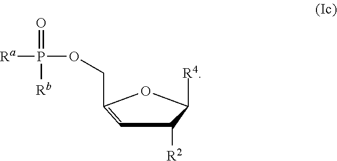

36-38. (canceled)

39. The compound of claim 1, which is selected from the group consisting of: 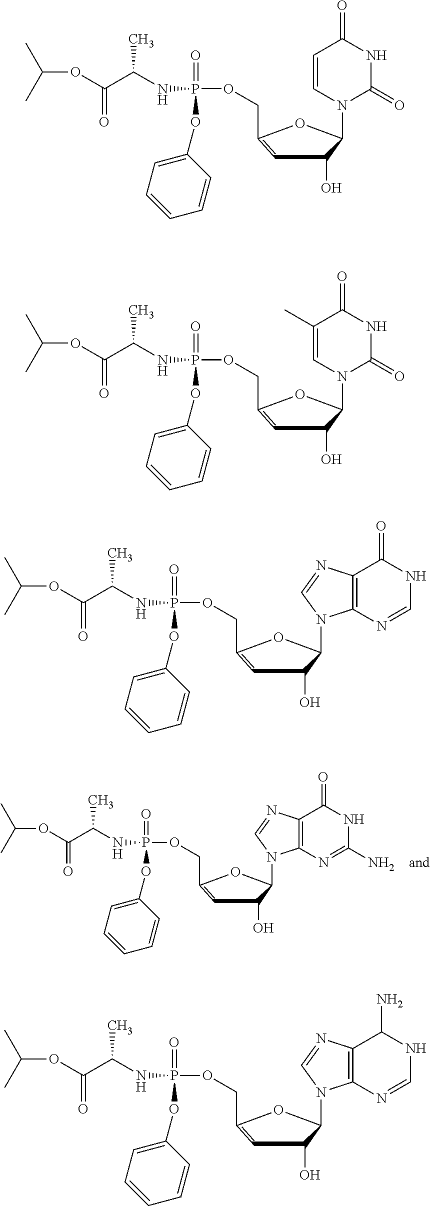
or a salt thereof.

40. A compound selected from the group consisting of: 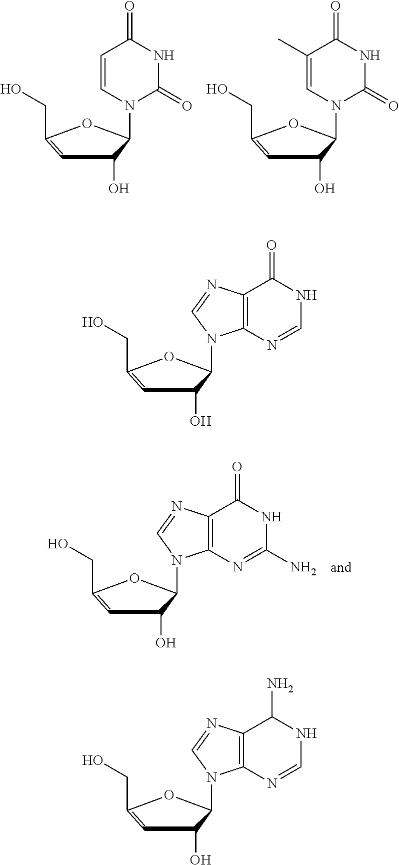
or a salt thereof.

41. A compound selected from the group consisting of: 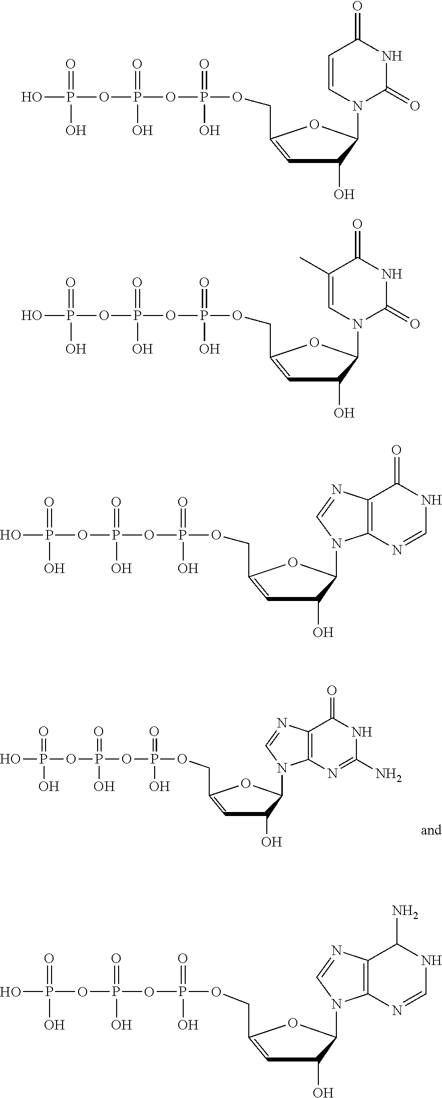
or a salt thereof.

42. A pharmaceutical composition comprising a compound as described in claim 1 or a pharmaceutically acceptable salt thereof and a pharmaceutically acceptable excipient.

43. A method for treating a viral infection in an animal comprising administering a compound of formula I as described in claim 1 or a pharmaceutically acceptable salt thereof to the animal.

44-46. (canceled)
